(19) United States
(12) Patent Application Publication
(10) Pub. No.: US 2006/0084630 A1
(43) Pub. Date: Apr. 20, 2006

(54) Method for synthesis of beta glucans

(76) Inventors: Harry E. Ensley, River Ridge, LA (US); David L. Williams, Johnson City, TN (US); Hai Yu, Davis, CA (US)

Correspondence Address: DONNA J. RUSSELL, 1492 ANTHONY WAY, MT. JULIET, TN, 37122, US

(21) Appl. No.: 11/244,639

(22) Filed: Oct. 6, 2005

Related U.S. Application Data
(60) Provisional application No. 60/616,869, filed on Oct. 7, 2004.

(51) Int. Cl. A61K 31/716; C07H 13/02

(52) U.S. Cl. 514/54; 536/119

(57) ABSTRACT

The invention provides a method for forming P-glucoside linkages in carbohydrate polymers, particularly synthetic glucan molecules, by attaching a protecting group to the C2 position of a glucoside donor. The protecting group of the invention stabilizes the dioxolenium ion and prevents orthoester formation to promote the β linkage and inhibit the undesirable a linkage. One such protecting group is 4-acetoxy-2,2-dimethylbutanoyl chloride.

This application claims the benefit of priority of earlier-filed U.S. provisional patent application No. 60/616,869, filed on Oct. 7, 2004.

### STATEMENT OF GOVERNMENT RIGHTS

This invention was developed in part with funding from the United States National Institutes of Health (NIH grant GM 53522). The U.S. government may therefore have certain rights in this invention.

### FIELD OF THE INVENTION

The present invention relates to methods for synthesis of polymers, such as polymers of glucose (e.g., anhydroglucose) units. The invention also relates to methods for forming β-linkages between units of a carbohydrate polymer.

### BACKGROUND OF THE INVENTION

Beta glucan, or β-1,3-linked polyglucose, comprises a family of molecules that are polymers of anhydroglucose repeat units forming a (1→3)-β-D-linked linear backbone with a glycosidic linkage between the 1- and 3-positions of the glucose units. They are major constituents of fungal cell walls. Common sources include, for example, medicinal mushrooms such as Sclerotium glucanicum, Lentinus edodes, and Schizophyllum commune, as well as Baker's yeast (Saccharomyces cerevisiae).

Water-soluble glucans tend to be semi-flexible single helices or triple helices, the triple helix being a complex of three intertwined single helices that are stabilized by extensive hydrogen bonding involving the C-2 hydroxyl group, located at the center of the helix.

β-glucans are important immunomodulators. β-glucan specific receptors have been discovered on primary cultures of normal human dermal fibroblasts, normal human vascular endothelial cells, human epithelial cells, human anterior pituitary cells, macrophages, and dendritic cells, for example. Glucan administration has been shown to increase resistance to a variety of infections, including gram-negative, gram-positive, fungal, viral, and parasitic infections. Pre- or post-treatment with glucan also improves survival outcome in polymicrobial sepsis. Glucans have been shown to prevent cardiac injury in response to ischemia/reperfusion. Topical or systemic administration of glucan enhances wound healing by increasing macrophage infiltration into the tissue proximal to a wound site, stimulating tissue granulation, collagen deposition, and renewal of epithelial tissue. A β-glucan/collagen preparation has also demonstrated therapeutic effect for the treatment of partial thickness burns in pediatric patients.

Dectin-1 has been identified as a glucan receptor which recognizes (1→3)-β- and (1→6)-β-linked glucans, also recognizing intact S. cerevisiae and C. albicans in a glucan-dependent fashion. Dectin is found on a variety of cells, with higher expression levels found on monocytes and neutrophils in blood, bone marrow, and spleen, as well as alveolar and inflammatory macrophages.

Glucans are presently sold as “natural products” isolated from cell walls of various microorganisms. Isolation procedures are based upon the limited solubility, or insolubility, of β-glucans in water. In most extraction protocols, cell walls are exhaustively extracted with dilute acid and base, and washed with alcohol to extract everything except the β-glucan. Standard isolation protocols, however, yield limited amounts of the polymer, with molecular weight distributions ranging from about 10³ to about 10⁶ grams/mole. Furthermore, the structure of glucans obtained by these methods varies, depending upon the source of the cell walls and the isolation procedure.

To provide a consistent supply of this therapeutically important biomolecule, it would be advantageous to produce β-glucan by synthetic methods. Current protocols for synthesis, however, provide molecules having a mixture of alpha and beta linkages. This is a problem because the biological effects of glucan are mediated by their interaction with the membrane receptors that have been shown to specifically recognize (1→3)-β-glucans, but generally do not recognize or bind α-linked glucosides.

What is needed, then, is a method for synthesizing such carbohydrate polymers having predominantly or exclusively beta glucoside linkages.

### SUMMARY OF THE INVENTION

The present invention relates to a method for forming β-glucoside linkages between carbohydrate units during synthesis of a carbohydrate polymer, the method comprising attaching a C2-carboxy protecting group to a glucoside donor to stabilize the intermediate dioxolenium ion and prevent orthoester formation.

In certain embodiments, the carbohydrate polymer may be a β-1,3-D-glucan or a β-1,6-D-glucan, for example and the carbohydrate units may be formed of one or more anhydroglucose units.

In one embodiment, the method for forming β-glucoside linkages between carbohydrate units in a polymer comprises attaching a protecting group to the C2 position of a glucoside donor, the protecting group having a general structure as in Examples A 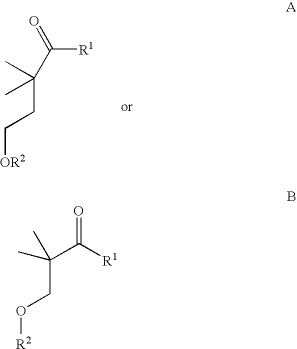
and B as shown, where R¹ is an activating group that facilitates ester formation and R², when removed, facilitates lactone formation and removal of the protecting group. In one embodiment, R¹ is optionally substituted with Cl. In another embodiment, R₁ is optionally substituted with anhydride.

In alternate embodiments, R² is optionally substituted with silane, acetate or other carboxylate ester.

In one embodiment, the protecting group is 4-acetoxy-2,2-dimethylbutanoyl chloride (ADMB).

The invention also provides carbohydrates, such as β-glucan molecules (e.g., β-1,3-D-glucan or β-1,6-D-glucan molecules) synthesized by the method, as well as pharmaceutical preparations comprising at least one β-1,3-D-glucan synthesized by the method of forming β-linkages between anhydroglucose molecules by attaching 4-acetoxy-2,2-dimethylbutanoyl chloride to a glucoside donor molecule prior to attachment of the donor to an acceptor molecule.

### BRIEF DESCRIPTION OF THE DRAWINGS

FIG. 1 illustrates formation of polymers having β-glucoside linkages or, alternately, formation of orthoesters (which can be intermediates in production of α-linked polymers), using standard synthesis methods known to those of skill in the art.

FIG. 2 is a diagram illustrating the structures of two synthetic molecules, a branched decasaccharide (FIG. 2a) and a linear decasaccharide (FIG. 2b), formed by the method of the present invention.

FIG. 3 is a diagram of the chemical synthesis of a thioglycoside 14 and an imidate 24 to provide units for synthesis of glucan molecules according to the method of the invention.

FIG. 4 illustrates reaction steps for formation of an acceptor molecule 30 for use in forming glucan molecules according to the method of the invention.

FIG. 5 illustrates reaction steps for formation of a thioglycoside molecule 40 having a C2-ADMB protecting group, which can be used in the method of the invention for forming a glucan molecule.

FIG. 6 describes reaction chemistry involved in forming a disaccharide molecule 42 having an ADMB protecting group, the disaccharide being useful for forming glucan molecules by the method of the invention.

FIG. 7 illustrates the chemical structure of two building blocks for glucan synthesis, a donor thiodisaccharide (“Thiodisaccharide 1” 42) and an acceptor disaccharide (“Disaccharide 2” 44).

FIG. 8 describes a reaction in which an acceptor molecule 30 is combined with a thioglycoside (Thioglycoside 1→4) to produce an acceptor disaccharide 44 after desilylation of intermediate 46.

FIG. 9 illustrates reaction steps for producing a donor trisaccharide 48 comprising a β-1,3-glucoside with a single β-1,6-glucoside linkage at the reducing terminus.

FIG. 10 through FIG. 21 illustrate reaction steps, as described herein, for preparing a linear decasaccharide and a branched decasaccharide glucan molecule as shown in FIG. 2a and FIG. 2b, according to the method of the present invention.

FIG. 22 is a graph illustrating the percentage of binding (Y axis) to the glucan-specific receptor, Dectin-1, of various concentrations (X axis) of two synthetic glucan molecules formed by the method of the present invention. Binding curves for the linear decasaccharide and branched decasaccharide are indicated by the symbols indicated on the graph.

### DETAILED DESCRIPTION

The inventors have developed a novel method for synthesizing carbohydrate polymers having beta linkages between the subunits. The method can be used to produce glucan molecules, for example, having predominantly or exclusively β linkages connecting the anhydroglucose units. As with many synthetic methods, chemically linking glucose molecules involves the use of protecting groups and removal of those groups to produce the final product molecules. The inventors have discovered that attachment of a C2-carboxy-protecting group to a glucoside donor, to stabilize the intermediate dioxolenium ion and prevent orthoester formation, can produce predominantly or exclusively β linkages and inhibit formation of the undesirable a linkage. In one embodiment, the protecting group is 4-acetoxy-2,2-dimethylbutanoyl chloride (ADMB).

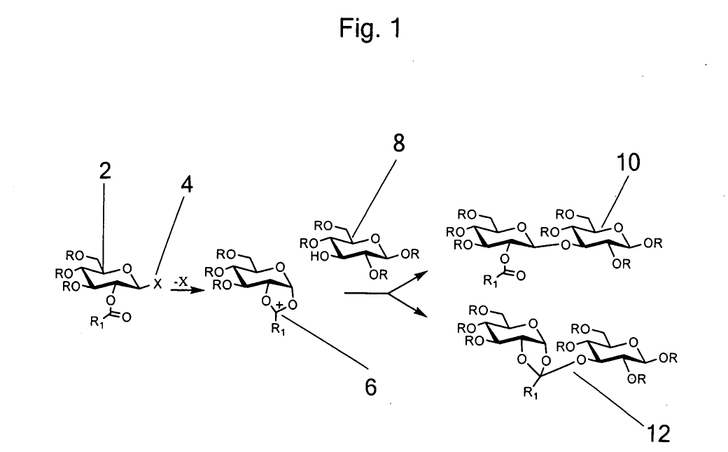

A method for synthesizing carbohydrates using sugar imidates as glycosyl donors and unprotected or partially protected glycosides as glycosyl receptors has previously been described. This method, however, produces both alpha (α) and beta (β) linkages. Stereoselective preparation of β-glucoside linkages are more reliably formed using a 2-carboxyprotected (“disarmed”) glucosyl donor, as shown in FIG. 1. Neighboring group participation of the 2-carboxy group of the donor 2 aids departure of the leaving group 4 to give the intermediate 6, which should produce the β-disaccharide 10 upon nucleophilic attack of the glucosyl acceptor 8.

Orthoester 12 formation, however, is an alternative outcome, rather than formation of the β-disaccharide. Since the orthoester can be an intermediate in the formation of the α-glycoside, as well as the β-disaccharide, orthoester formation is undesirable when the goal is production of exclusively β-linked molecules. The inventors provide here a method for stabilizing the dioxolenium ion and sterically hindering orthoester formation so that the reaction will yield the β-glycoside.

The inventors and others have previously utilized various protecting groups, some of which are listed in Table 1, with varying results, as shown.

TABLE 1
Glycosylation using glycosyl donors with various ester-protecting groups at C2.

| Donor | Acceptors | |
|---|---|---|
| 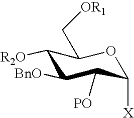 | 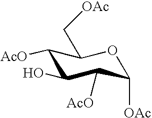 | 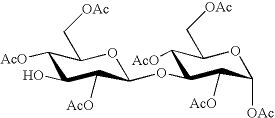 |
| P = Ac | α:β 1:1 84% | NA |
| X = O-trichloroacetimidate^{b} | | |
| P = Bz | NA | α only 86% |
| X = O-trichloroacetimidate^{b} | | |
| P = ClCH₂CO | NA | α only 85% |
| X = O-trichloroacetimidate^{b} | | |
| P = CH₃OCO | α:β 1:1 85% | α only 83% |
| X = O-trichloroacetimidate^{b} | | |
| P = (CH₃)₃CCO | β only 80% | α:β 2:3 81% |
| X = O-trichloroacetimidate^{b} | | |
| P = (CH₃)₃CCO | NA | α:β 1:3 82% |
| X = SPh^{c} | | |

R₁ = Ac, R₂ = Ac, except for X = SPh, where R₁:R₂ = CHPh.
^{b}Activation with TMSOTf.
^{c}Activation with NIS/AgOTf

Glycosylation using glycosyl donors with the ester protecting groups listed in Table 1 with 1,2,4,6-tetra-O-acetyl-α-D-glycopyranose or the β-linked disaccharide produced molecules with glycosidic linkage ranging from β-only to α-only.

As shown in Table 1, formation of the undesirable α-linkage occurs both for trichloroacetimidate activation and for thioglycoside mediated couplings. The use of pivaloate esters at C2 (C2-Piv) aids formation of the β-glycoside but does not completely inhibit formation of the α-glycoside. Although there are stereochemical benefits of the C2-Piv group the inventors were concerned that the harsher conditions required for deprotection of multiple pivaloate esters at sterically hindered secondary centers would be a limitation for 1,3-β-glucan production. Therefore, they endeavored to develop a protecting group to provide the steric advantages of the bulky pivaloyl group but without the need to use harsh agents to remove the protecting group. They found that treatment of methyl 4,6-benzylidene-α-D-glucoside and ethyl 4,6-benzylidene-1-thio-α-D-glucoside with 4-acetoxy-2,2-dimethylbutanoyl chloride (ADMB) permits selective acylation of the C2 hydroxyl group in 95 and 94% yield, respectively. The ease of removal of the ADMB ester group on treatment with catalytic quantity of diazabicycloundecane (DBU) in methanol at room temperature is demonstrated in Table 2.

TABLE 2
Comparison of Hydrolysis of Pivaloyl and 4-acetoxy-2,2-dimethylbutanoyl
esters(ADMB)

| substrate | | equiv of DBU | time (h) | ROH yield |
|---|---|---|---|---|
| 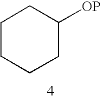 | 4a P = Piv 4b P = ADMB 4b | 0.5 0.5 0.1 | 24 0.5 2 | 0 100% 100% |
| 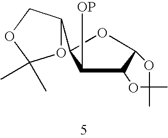 | 5a P = Piv 5b P = ADMB 5b | 0.5 0.5 0.1 | 10 1 3 | 12% 100% 100% |
| 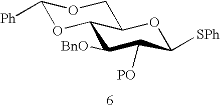 | 6a P = Piv 6b P = ADMB | 0.5 0.1 | 24 3 | 0 98% |
| 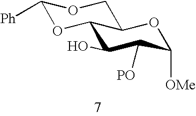 | 7a P = Piv 7a 7b P = ADMB | 0.5 0.5 0.5 | 2 12 1 | 30% 97% 99% |

Alpha-glycoside formation is minimal or non-existent when using the C2-ADMB group in carbohydrate coupling reactions. Couplings formed using C2-ADMB give 80-90% yields of β-glycoside with only traces of the α-anomer detectable by thin layer chromatography (TLC). For example, as indicated in Table 3, coupling the glycoside and disaccharide indicated in row 1 gave an 83% yield of the all β-linked trisaccharide as the only product when ADMB was used as the protecting group.

TABLE 3
Glycosylation using glycosyl donors with C2 4-Acetoxy-2,2-
dimethylbutanoate protecting group.

| Donor | Acceptor | Product |
|---|---|---|
| 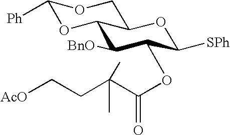 | 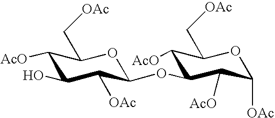 | 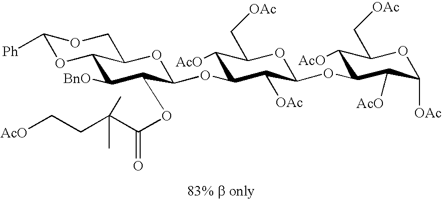 |
| 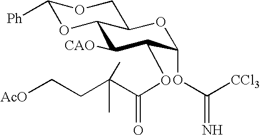 | 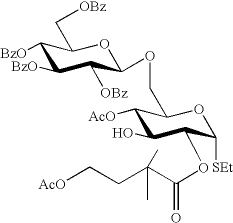 | 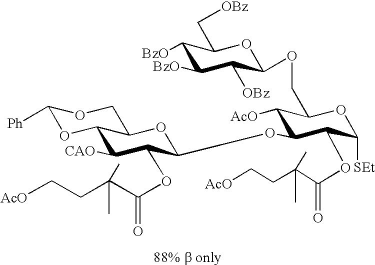 |
| 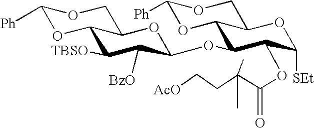 | 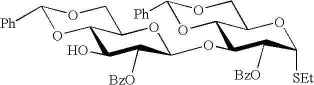 | 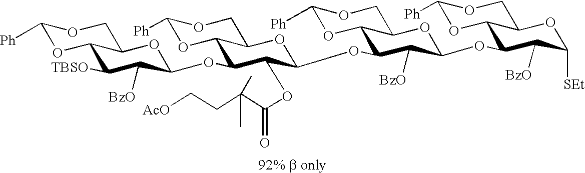 |
| 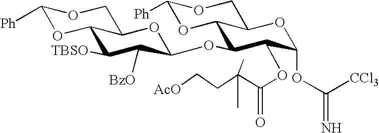 | 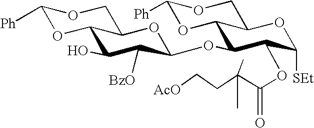 | 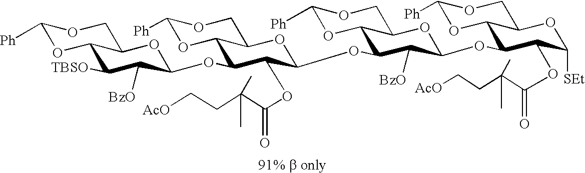 |

High yields of β-glycosides are obtained in all of the cases the inventors have examined. The presence of the ADMB group may stabilize the dioxolenium ion 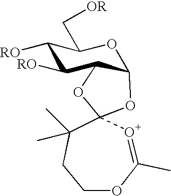
and sterically prevent orthoester formation (the orthoester being an intermediate in the formation of the α-glycoside).

ADMB has previously been described as an intermediate formed during the synthesis of a macrocyclic lactone HMG-CoA reductase inhibitor. Synthesis of ADMB is described in U.S. Pat. No. 4,665,091 (Hoffman), at column 7, lines 30 through 50. The inventors have discovered that this compound provides an effective protecting group for the synthesis of complex carbohydrates, especially glucan molecules, having predominantly or exclusively beta linkages between the glucose units.

The invention therefore also provides a method for synthesizing β-glucosides using protecting groups, especially C2-carboxy protecting groups, such as, for example, 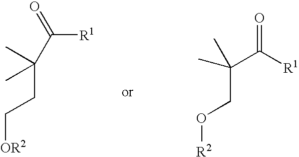
where R¹ is an activating group that facilitates ester formation and R² is a protecting group that, when removed, facilitates lactone formation and removal of the ADMB group. R¹ may be optionally substituted with, for example, Cl or anhydride and R² may be optionally substituted with, for example silane or acetate. The inventors have shown that these types of molecules are particularly effective for synthesizing β-glucan molecules having predominantly or exclusively beta linkages between the monosaccharide (e.g., anhydroglucose) units.

The method of the invention can be used to form a variety of carbohydrate polymers having beta linkages. The following non-limiting examples describe syntheses of a branched and an unbranched (linear) glucan decasaccharide according to the method of the present invention.

### EXAMPLES

N-bromosuccinimide (NBS), Trimethylsilyltriflate (TMSOTf), t-Butyldimethylsilyl chloride (TBDMSCl), Triethylamine (Net3), Dimethylformamide (DMF), Benzaldehydedimethylacetal, Ethyl mercaptan (EtSH), and Tin tetrachloride (SnCl4) were obtained from ACROS Organics (Fischer-Scientific). The remaining reagents used in the syntheses described below were obtained from Aldrich Chemical (Sigma-Aldrich, St. Louis, Mo.).

Synthesis of the Branched Decasaccharide and the Linear Decasaccharide

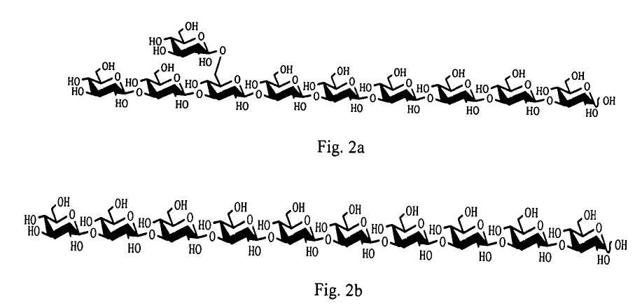

Synthesis of the branched decasaccharide shown in FIG. 2a and the and linear decasaccharide shown in FIG. 2b required building blocks that were assembled to give protected forms of the branched and linear decasaccharides. Removal of the protecting groups produced the decasaccharides shown in FIG. 2 a and b.

Four monosaccharides are important intermediates for glucan synthesis:
Imidate (donor):
O-[2-O-benzoyl-4,6-O-benzylidene-3-O-(t-butyldimethylsilyl)]-α-D-glycopyranosyl trichloroacetimidate; 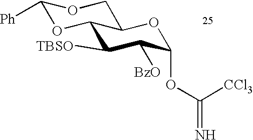
Two thioglycosides:
Ethyl 2-O-benzoyl-4,6-O-benzylidene-3-O-(t-butyldimethylsilyl)-1-thio-β-D-glycopyranoside (“Thioglycoside 1”) 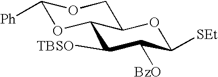
   and Ethyl 2-O-(4-acetoxy-2,2-dimethylbutanoyl)-4,6-O-benzylidene-1-thio-α-D-glycopyranoside (“Thioglycoside 2”); and 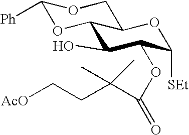
Acceptor:
Benzoyl 2-O-benzoyl-4,6-O-benzylidene-α-D-glycopyranoside 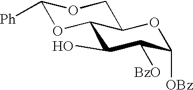
   Synthesis of the Imidate and Thioglycoside 1:

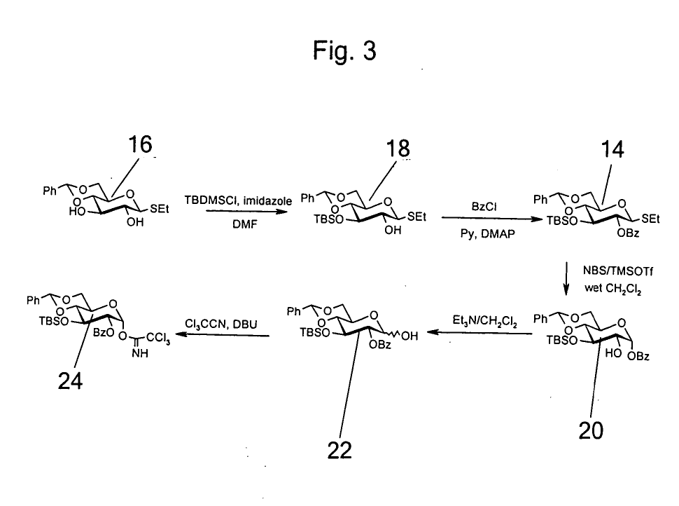

Briefly, imidate 24 and the thioglycoside 1 14 are prepared as illustrated in FIG. 3. To prepare thioglycoside 1 14, to a solution of Ethyl 4,6-O-benzylidene-1-thio-β-D-glycopyranoside 16 (10.19 g, 31.6 mmol) and imidazole (3.26 g, 47.4 mmol) in dimethylformamide (50 mL), t-butyldimethylsilylchloride (5.23 g, 34.7 mmol) was added in small portions at 0° C. The mixture was stirred at room temperature overnight and then diluted with EtOAc (300 ml) and the organic layer was washed with saturated ammonium chloride. The aqueous layer was extracted with EtOAc (100 ml). The combined organic layers were washed with saturated sodium bicarbonate and then brine. After the mixture was dried over Na₂SO₄, the solvent was evaporated and the residue was purified by silica gel column chromatography (hexane:EtOAc 12:1) to give Ethyl 4,6-O-benzylidene-3-O-(t-butyldimethylsilyl)-1-thio-β-D-glycopyranoside 18 (12.6 g, 90%) as a colorless oil.

To a solution of alcohol Ethyl 4,6-O-benzylidene-3-O-(t-butyldimethylsilyl)-1-thio-β-D-glycopyranoside 18 (4.47 g, 10.49 mmol) and dimethylaminopyridine (630 mg) in a mixture of pyridine (20 mL) and CH₂Cl₂ (30 mL), benzoyl chloride (3.04 mL) was added at 0° C. Then the mixture was heated to 80° C. and stirred overnight with protection from moisture with a calcium chloride packed tube. The solvent was removed in vacuo and the residue was taken up in EtOAc. The organic layer was washed with saturated ammonium chloride. The aqueous layer was back-extracted with EtOAc. The combined ethyl acetate layers were washed with saturated sodium bicarbonate and brine. The organic layer was dried over Na₂SO₄, and the solvent was evaporated. The residue was purified by silica gel column chromatography (hexane:EtOAc 14:1) to afford the benzoate Ethyl 2-O-benzoyl-4,6-O-benzylidene-3-O-(t-butyldimethylsilyl)-1-thio-β-D-glycopyranoside 14 (5.45 g, 98%). [H NMR δ 8.30-7.47 (m, 10H), 5.68 (s, 1H), 5.41 (dd, J=10.4 Hz and 8.4 Hz, 1H, H-2), 4.87 (d, J=10.4 Hz, 1H, H-1), 4.52 (dd, J=10.4 Hz and 4.8 Hz, 1H, H-6a), 4.18 (t, J=8.4 Hz, 1H, H-3), 3.93 (t, J=10.4 Hz, 1H, H-6b), 3.77 (t, J=9.6 Hz, 1H, H-4), 3.69 (m, 1H, H-5), 2.86 (m, 2H), 1.35 (t, 3H), 0.82 (s, 9H), 0.08 (s, 3H), 0.00 (s, 3H); ¹³C NMR δ 170.2, 142.0, 139.5, 138.1, 135.5, 134.8, 134.2, 133.8, 133.3, 133.1, 131.2, 106.8, 89.1, 86.4, 79.2, 78.2, 75.8, 73.6, 30.5, 23.0, 0.52, 0.00.]

To prepare the imidate, Ethyl 2-O-benzoyl-4,6-O-benzylidene-3-O-(t-butyldimethylsilyl)-1-thio-β-D-glycopyranoside 14 (500 mg, 0.94 mmol) was dissolved in 5 ml of CH₂Cl₂-H₂O (100:1) and cooled to 0° C. The solution was treated with N-bromosuccinimide (167 mg, 0.94 mmol, 1.0 equiv.) and 0.1 equiv. of TMSOTf. The resulting mixture was stirred at 0° C., and the reaction was monitored by TLC. After the reaction was complete, aqueous NaHCO₃ was added, and the reaction mixture was extracted with CH₂Cl₂ three times. The combined organic phase was washed with brine, dried over anhydrous Na₂SO₄, and concentrated by rotary evaporation. The organic layer was purified by flash column chromatography (hexane/EtOAc 8:1) to yield Benzoyl 4,6-O-benzylidene-3-O-(t-butyldimethylsilyl)-α-D-glycopyranoside 20 (412 mg, 90%) as a white amorphous solid. [H NMR δ 8.00-7.26 (m, 10H), 6.41(d, J=4.0 Hz, 1H, H-1), 5.46 (s, 1H), 4.20 (dd, J=10.4 Hz and 5.2 Hz, 1H, H-6a), 4.02 (t, J=9.2 Hz, 1H, H-3), 3.90 (m, 1H, H-5), 3.80 (dd, J=9.2 Hz and 4.0 Hz, 1H, H-2), 3.65 (t, J=10.4 Hz, 1H, H-6b), 3.50 (t, J=9.2 Hz, 1H, H-4), 0.81 (s, 9H), 0.06 (s, 3H), 0.00 (s, 3H); ¹³C NMR δ 169.9, 141.7, 138.4, 134.6, 134.0, 133.7, 133.3, 132.9, 130.9, 106.4, 97.3, 85.9, 77.5, 77.4, 73.4, 70.0, 30.6, 23.0, 0.51, 0.00.]

Benzoyl 4,6-O-benzylidene-3-O-(t-butyldimethylsilyl)-α-D-glycopyranoside 20 (400 mg, 0.82 mmol) was dissolved in a mixture of CH₂Cl₂-Et₃N (4:1, v/v, 5 mL). The solution was stirred at room temperature for 24 h. After the reaction was complete, the solution was concentrated and purified by flash column chromatography to give 2-O-benzoyl-4,6-O-benzylidene-3-O-(t-butyldimethylsilyl)-D-glycopyranose 22 (380 mg, 95%). [H NMR δ 8.21-7.35 (m, 10H), 5.64 (s, 1H), 5.23 (dd, J=9.2 Hz, 1H, H-2), 4.97 (d, J=9.2 Hz, 1H, H-1), 4.52 (dd, J=10.0 Hz and 3.2 Hz, 1H, H-6a), 4.23 (t, J=9.6 Hz, 1H, H-3), 3.88-3.66 (m, 3H, H-4, H-6b, H-5), 0.79 (s, 9H), 0.07 (s, 3H), 0.00 (s, 3H)]

To the solution of 2-O-benzoyl-4,6-O-benzylidene-3-O-(t-butyldimethylsilyl)-D-glycopyranose 22 (2.2 g, 4.5 mmol) in anhydrous CH₂Cl₂ (20 mL) was added trichloroacetonitrile (Cl₃CCN, 2.0 mL, 5.0 equiv.) and 1,8-diazabicyclo[5.4.0]undec-7-ene (DBU, 50 μL) at 0° C. After stirring the resulting mixture for 4 h, the solvent was evaporated under reduced pressure and the resulting oil was purified by flash column chromatography to give pure 0-[2-O-benzoyl-4,6-O-benzylidene-3-O-(t-butyldimethylsilyl)]-α-D-glycopyranosyl trichloroacetimidate 24 (2.71 g, 95%) as a white amorphous solid. [H NMR δ 8.57 (s, 1H, NH), 8.09-7.39 (m, 10H), 6.62 (d, J=4.0 Hz, 1H, H-1), 5.65 (s, 1H), 5.42 (dd, J=9.6 Hz and 4.0 Hz, 1H, H-2), 4.48 (t, J=9.6 Hz, 1H, H-3), 4.21 (dd, J=10.4 Hz and 4.8 Hz, 1H, H-6a), 4.15 (m, 1H, H-5), 3.85 (t, J=10.4 Hz, 1H, H-6b), 3.78 (t, J=9.6 Hz, 1H, H-4), 0.80 (t, 9H), 0.08 (s, 3H), 0.02 (s, 3H).]

Synthesis of the Acceptor

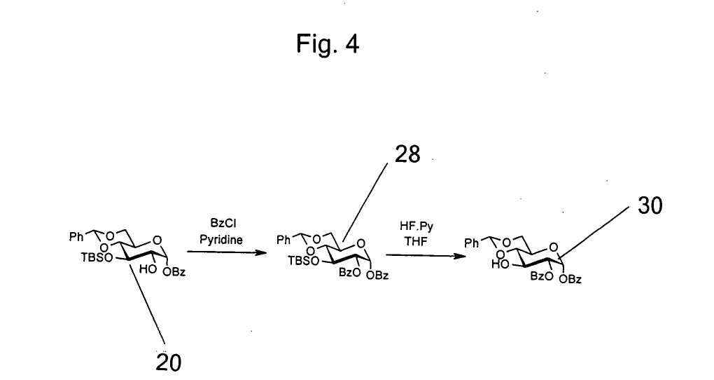

The acceptor molecule 30 is synthesized according to reaction chemistry illustrated in FIG. 4. Briefly, to a stirred solution of compound Benzoyl 4,6-O-benzylidene-3-O-(t-butyldimethylsilyl)-α-D-glycopyranoside 20 (4 g, 8.22 mmol) in dry pyridine (15 mL) was added benzoyl chloride(1.6 mL). After stirring overnight at room temperature, the mixture was diluted with CH₂Cl₂ (50 mL) and washed with a dilute hydrochloric acid solution, a saturated sodium bicarbonate solution, and then brine. The organic phase was dried over anhydrous MgSO₄ and evaporated to dryness. The residue was purified by silica gel column chromatography to afford Benzoyl 2-O-benzoyl-4,6-O-benzylidene-3-O-(t-butyldimethylsilyl)-α-D-glycopyranoside 28 (4.8 g, 99%). [H NMR δ 8.13-7.28 (m, 15H), 6.67 (d, J=4.0 Hz, 1H, H-1), 5.66 (s, 1H), 5.49 (dd, J=9.6 Hz and 4.0 Hz, 1H, H-2), 4.49 (t, J=9.2 Hz, 1H, H-3), 4.18 (dd, 1H, H-6a), 4.15 (m, 1H, H-5), 3.96-3.79 m, 2H, H-4, H-6b), 0.72 (t, 9H), 0.04 (s, 3H), 0.00 (s, 3H).]

To a stirred solution of Benzoyl 2-O-benzoyl-4,6-O-benzylidene-3-O-(t-butyldimethylsilyl)-α-D-glycopyranoside 28 (4.5 g, 7.62 mmol) in dry THF (20 mL) was added hydrofluoric acid-pyridine complex (1.5 mL) at 0° C. under Argon. After stirring for 24 h at room temperature, TLC showed that the starting material had disappeared. The mixture was diluted with ether, washed with aqueous sodium bicarbonate and brine. The organic phase was dried over anhydrous MgSO₄ and evaporated to dryness. The crude product was subjected to flash column chromatography to give Benzoyl 2-O-benzoyl-4,6-O-benzylidene-α-D-glycopyranoside 30 (3.34 g, 92%) as a white amorphous solid. [H NMR δ 8.08-7.24 (m, 15H), 6.66 (d, J=4.0 Hz, 1H, H-1), 5.62 (s, 1H), 5.40 (dd, J=9.6 Hz and 4.0 Hz, 1H, H-2), 4.15 (dd, J=9.2 Hz, 1H, H-3), 4.18 (dd, 1H, H-6a), 4.15 (m, 1H, H-5), 3.96-3.79 (m, 2H, H-4, H-6b).]

Preparation of Thioglycoside 2 with ADMB Protecting Group

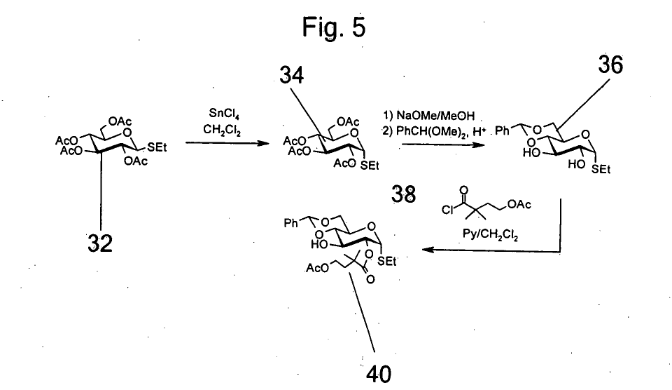

Thioglycoside 2 is synthesized as illustrated in FIG. 5. To a stirred solution of Ethyl 2,3,4,6-tetra-O-acetyl-1-thio-β-D-glycopyranoside 32 (80 g, 0.20 mol) in anhydrous CH₂Cl₂ (700 mL) at 0° C. was dropwise added SnCl₄ (23.8 mL, 1.0 equiv.), and the mixture was stirred for 15 h at room temperature (rt) under a nitrogen atmosphere. The mixture was poured into ice-H₂O, and the organic layer was separated, washed successively with aqueous sodium bicarbonate, H₂O and brine, dried, and concentrated. The resulting solid was crystallized from Et₂O-hexane and recrystallized twice from ethanol to give Ethyl 2,3,4,6-tetra-O-acetyl-1-thio-α-D-glycopyranoside 34 (36 g, 45%). [H NMR δ 5.68 (d, J=5.6 Hz, 1H, H-1), 5.31 (dd, J=10.0 and 9.6 Hz, 1H, H-4), 5.08-4.98 (m, 2H, H-2, H-3), 4.47-4.40 (m, 1H, H-5), 4.30 (dd, J=12.4 and 4.6 Hz, 1H, H-6a), 4.07 (dd, J=12.4 and 2.2 Hz, 1H, H-6b), 2.63-2.48 (m, 2H), 2.08 (s, 3H), 2.06 (s, 3 H), 2.03 (s, 3H), 2.01 (s, 3H), 1.26 (t, J=7.4 Hz, 3H).]

To a solution of thioglycoside Ethyl 2,3,4,6-tetra-O-acetyl-1-thio-α-D-glycopyranoside 34 (35 g, 127 mmol) in anhydrous CH₂Cl₂/MeOH (1:1; 300 mL) was added a catalytic quantity of sodium methoxide (NaOMe, 10 mg) at room temperature and the reaction mixture was stirred overnight. The mixture was neutralized with Dowex 50W-X8 (H+), filtered, and the filtrate was concentrated in vacuo. The residue was dried by sequentially adding and evaporating toluene (3×30 mL). To a solution of the residue in dimethylformamide (200 mL) was added PhCH(OMe)₂ (23 mL, 152 mmol) and TsOH.H₂O (200 mg) at room temperature. The reaction mixture was warmed to 50° C. under reduced pressure (15 mmHg) and then stirred for 2 h. The reaction was quenched by addition of Et₃N (5 ml) and diluted with AcOEt (300 ml). The solution was washed with water and brine, dried over MgSO₄, and concentrated in vacuo. The residue was recrystallized from CH₂Cl₂: hexane to give Ethyl 4,6-O-benzylidene-1-thio-α-D-glycopyranoside 36 as a white solid (34 g, 86%).

To a stirred solution of compound Ethyl 4,6-O-benzylidene-1-thio-α-D-glycopyranoside 36 (1.71 g, 5.48 mmol) in pyridine (15 mL) at 0° C. was slowly added 4-acetoxy-2,2-dimethylbutanoyl chloride 38(1.2 g). The mixture was stirred overnight at room temperature. The reaction mixture was concentrated and then diluted with ethyl acetate and washed with a dilute hydrochloric acid solution, saturated aqueous sodium bicarbonate, and then brine. The organic phase was dried over anhydrous MgSO₄ and evaporated to dryness. The crude product was subjected to flash column chromatography to afford Ethyl 2-O-(4-acetoxy-2,2-dimethylbutanoyl)-4,6-O-benzylidene-1-thio-α-D-glycopyranoside 40 (2.3 g) as white solid. [H NMR δ 7.50-7.32 (m, 5H), 5.60 (d, J=6.4 Hz, 1H, H-1), 5.54 (s, 1H), 4.90 (dd, J=9.6 Hz and 6.0 Hz, 1H, H-2), 4.29-4.17 (m, 3H, H-6a, CH₂), 4.10 (t, J=9.6 Hz, 1H, H-3), 4.02 (m, 1H, H-5), 3.76 (t, J=10.4 Hz, 1H, H-6b), 3.58 (t, J=9.6 Hz, 1H, H-4), 2.58-2.46 (m, 2H), 2.06-2.00 (m, 1H), 1.99 (s, 3H), 1.82-1.75 (m, 1H), 1.26 (s, 3H), 1.23 (t, J=7.2 Hz, 3H), 1.21 (s, 3H).]

Preparation of Disaccharides

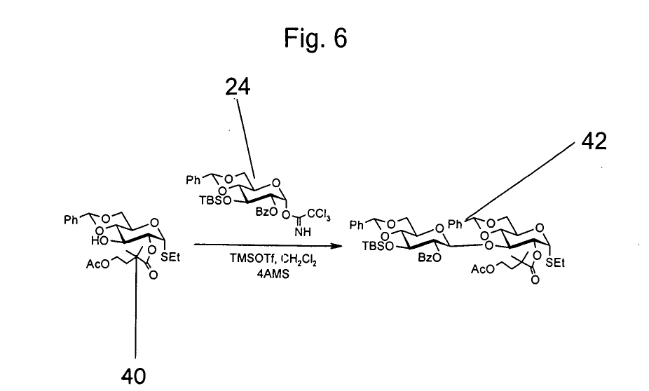

From the monosaccharides, two important disaccharides were synthesized without using the ADMB protecting group. Using reaction chemistry shown in FIG. 6, thioglycoside 2 was combined with the imidate donor to produce thiodisaccharide 1 (S-Ethyl 2-O-(4-acetoxy-2,2-dimethylbutanoyl)-4,6-O-benzylidene-3-O-[2-O-benzoyl-4,6-O-benzylidene-3-O-(t-butyldimethylsilyl)]-1-thio-α-D-glycopyranoside) 42 (FIG. 6).

To a stirred mixture of Thioglycoside 2 (Ethyl 2-O-(4-acetoxy-2,2-dimethylbutanoyl)-4,6-O-benzylidene-1-thio-α-D-glycopyranoside) 40 (916 mg, 1.95 mmol), imidate (0-[2-O-benzoyl-4,6-O-benzylidene-3-O-(t-butyldimethylsilyl)]-α-D-glycopyranosyl trichloroacetimidate) 24 (1.35 g, 1.1 eq.), and molecular sieves (4 Å, 1.5 g) in CH₂Cl₂ (20 mL) at −70° C. was added TMSOTf (0.1 N in CH₂Cl₂, 1.95 mL). After stirring for 6 h, the reaction mixture was quenched with Et₃N (0.2 mL), filtered, and the filtrate was concentrated in vacuo. The residue was purified by silica gel column chromatography to give Thiodisaccharide 1 (S-Ethyl 2-O-(4-acetoxy-2,2-dimethylbutanoyl)-4,6-O-benzylidene-3-O-[2-O-benzoyl-4,6-O-benzylidene-3-O-(t-butyldimethylsilyl)]-1-thio-α-D-glycopyranoside) 42 (1.68 g, 92%). [H NMR δ 8.01-7.40 (m, 15H), 5.76 (d, J=5.6 Hz, 1H, H-1), 5.69 (s, 1H), 5.34 (s, 1H), 5.33 (t, J=7.2 Hz, 1H, H-2′), 5.16 (d, J=7.2 Hz, 1H, H-1′), 5.03 (dd, J=9.6 Hz and 6.0 Hz, 1H, H-2), 4.46-4.36 (m, 4H), 4.25-4.20 (m, 2H), 4.03 (dd, J=8.3 Hz and 7.4 Hz, 1H, H-3′), 3.93 (t, J=9.6 Hz, 1H), 3.87-3.81 (m, 3H), 3.56 (m, 1H), 2.63 (m, 2H, CH₂), 2.16 (s, 3H), 2.02-1.98 (m, 2H), 1.34 (t, J=7.6 Hz, 3H), 1.30 (s, 3H), 1.29 (s, 3H), 0.85 (s, 9H), 0.06 (s, 3H), 0.00 (s, 3H); ¹³C NMR δ 176.3, 171.2, 165.2, 137.4, 137.4, 133.3, 130.1, 130.0, 129.5, 129.1, 128.6, 128.5, 128.3, 126.5, 126.4, 102.0, 101.6, 99.5, 82.3, 81.3, 79.8, 75.7, 74.1, 73.8, 73.3, 68.9, 66.3, 62.9, 61.6, 40.9, 38.1, 25.8, 25.2, 25.2, 24.4, 21.3, 18.1, 15.0, −4.05, −4.76.]

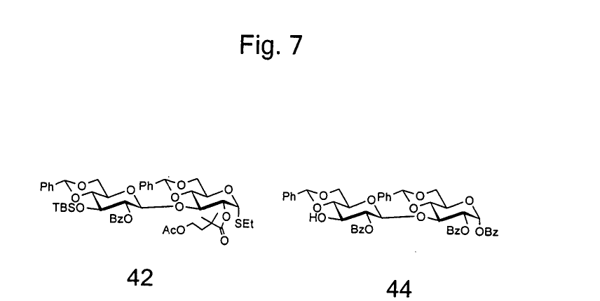

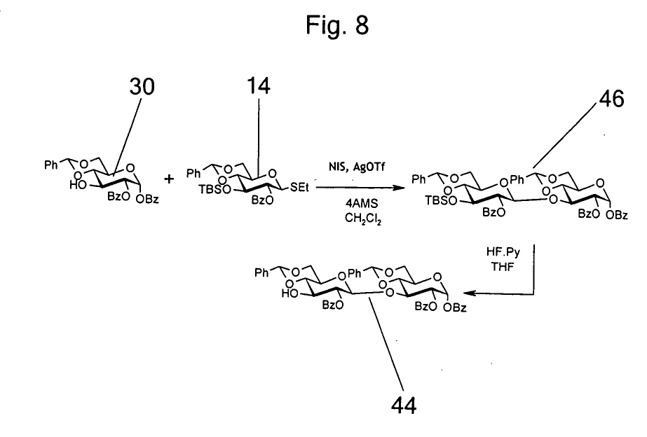

Disaccharide 2 (Benzoyl 2-O-benzoyl-4,6-O-benzylidene-3-O-[2-O-benzoyl-4,6-O-benzylidene-β-D-glycopyranosyl]-α-D-glycopyranoside) 44 (FIG. 7) was produced by coupling the acceptor 30 with thioglycoside 1 14 to give the disaccharide Benzoyl 2-O-benzoyl-4,6-O-benzylidene-3-O-[2-O-benzoyl-4,6-O-benzylidene-3-O-(t-butyldimethylsilyl)-β-D-glycopyranosyl]-α-D-glycopyranoside 46, from which the TBS group was cleaved by HF/Py to give disaccharide 2 44 (FIG. 8).

Briefly, to a stirred mixture of Benzoyl 2-O-benzoyl-4,6-O-benzylidene-α-D-glycopyranoside 30 (840 mg, 1.76 mmol), thioglycoside Ethyl 2-O-benzoyl-4,6-O-benzylidene-3-O-(t-butyldimethylsilyl)-1-thio-β-D-glycopyranoside 14 (1.13 g, 2.13 mmol), and molecular sieves (4 Å, 1.5 g) in CH₂Cl₂ (20 mL) at −20° C. were added N-iodosuccinimide (660 mg) and silver triflate (100 mg, in 1 ml dry toluene). After stirring for 2 h, the reaction mixture was quenched with Et₃N (0.05 mL), filtered, and concentrated in vacuo. The residue was purified by silica gel column chromatography using hexanes and ethyl acetate (4:1→2:1, v/v) to give Benzoyl 2-O-benzoyl-4,6-O-benzylidene-3-O-[2-O-benzoyl-4,6-O-benzylidene-3-O-(t-butyldimethylsilyl)-β-D-glycopyranosyl]-α-D-glycopyranoside 46(1.46 g, 88%) as a white amorphous solid.

To a stirred solution of 46 (2.1 g, 2.22 mmol) in dry THF (20 mL) was added hydrofluoric acid-pyridine complex (1 mL) at 0° C. under argon. After stirring for 24 h at room temperature, TLC showed that the starting material had disappeared. The mixture was diluted with ether, washed with aqueous sodium bicarbonate and brine. The organic phase was dried over anhydrous MgSO₄ and evaporated to dryness. The crude product was subjected to flash column chromatography using hexanes and ethyl acetate (2:1, v/v) as eluant to afford the pure product Benzoyl 2-O-benzoyl-4,6-O-benzylidene-3-O-[2-O-benzoyl-4,6-O-benzylidene-β-D-glycopyranosyl]-α-D-glycopyranoside 44 (1.68 g, 91%) as a white amorphous solid.

Preparation of a Branched Glucan

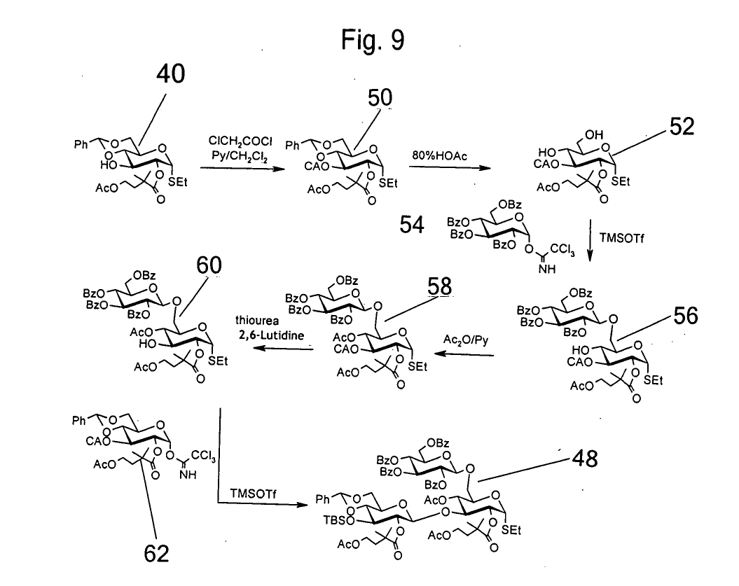

A disaccharide that can add the β-1,6 branched glycosidic bond to a synthetic glucan molecule was synthesized and used to produce a branched trisaccharide (Ethyl 2-O-(4-acetoxy-2,2-dimethylbutanoyl)-3-O-[2-O-benzolyl-4,6-O-benzylidene-3-O-3-O-chloroacetyl-β-D-glycopyranosyl]-4-O-acetyl-6-O-[2,3,4,6-tetra-O-benzoyl)-β-D-glycopyranosyl]-1-thio-α-D-glycopyranoside) 48 with β-1,3 and β-1,6 linkages as shown in FIG. 9.

Briefly, chloroacetyl chloride (70 μL, 0.88 mmol) was added to a cooled (0° C.) solution of Ethyl 2-O-(4-acetoxy-2,2-dimethylbutanoyl)-4,6-O-benzylidene-1-thio-α-D-glycopyranoside 40 (0.4 g, 0.84 mmol) in CH₂Cl₂ (30 mL) and pyridine (1.5 mL). After stirring for 2 h, toluene (50 ml) was added and the mixture was concentrated in vacuo, diluted with CH₂Cl₂, and washed with 10% (w/v) aqueous NaCl. The organic layer was dried, filtered, and concentrated. The residue was purified by column chromatography to afford S-Ethyl 2-O-(4-acetoxy-2,2-dimethylbutanoyl)-4,6-O-benzylidene-3-O-chloroacetyl-1-thio-α-D-glycopyranoside 50 (0.45 g, 98%). [H NMR δ 7.99-7.24 (m, 5H), 5.78 (t, J=10.0 Hz, 1H, H-4), 5.71 (d, J=6.0 Hz, 1H, H-1), 5.53 (s, 1H), 5.13 (dd, J=10.0 Hz and 5.6 Hz, 1H, H-2), 4.23 (m, 1H, H-5), 4.29 (dd, J=10.4 Hz and 4.8 Hz, 1H, H-6a), 3.92-3.81 (m, 4H), 2.62-2.51 (m, 2H), 1.88 (s, 3H), 1.83-1.74 (m, 2H), 1.26 (t, J=7.6 Hz, 3H), 1.09 (s, 3H), 1.05 (s, 3H).]

A solution of S-Ethyl 2-O-(4-acetoxy-2,2-dimethylbutanoyl)-4,6-O-benzylidene-3-O-chloroacetyl-1-thio-α-D-glycopyranoside 50 (1.25 mg, 2.30 mmol) in 80% AcOH (10 mL) was heated to 80° C. for 3 h. The solution was evaporated in vacuo, passed through a short silica gel column and concentrated, the residue Ethyl 2-O-(4-acetoxy-2,2-dimethylbutanoyl)-3-O-chloroacetyl-1-thio-α-D-glycopyranoside 52 was directly used for the next step.

To a stirred mixture of the above monosaccharide residue, 2,3,4,6-tetra-O-benzoyl-α-D-glycopyranosyl trichloroimidate 54 (1.7 g, 2.30 mmol), and molecular sieves (4 Å, 2 g) in CH₂Cl₂ (30 mL) at −70° C. was added TMSOTf (0.1 N in CH₂Cl₂, 2.5 mL). After stirring for 8 h, the reaction mixture was quenched with Et₃N (0.2 mL), filtered, and the filtrate was concentrated in vacuo. The residue Ethyl 2-O-(4-acetoxy-2,2-dimethylbutanoyl)-3-O-chloroacetyl-6-O-[2,3,4,6-tetra-O-benzoyl)-β-D-glycopyranosyl]-1-thio-α-D-glycopyranoside 56 was directly used for the next step.

To the stirred solution of the above residue in dry pyridine (10 mL) was added Ac₂O (1 mL). After stirring overnight at room temperature, the mixture was diluted with CH₂Cl₂ (40 mL) and washed with dilute HCl solution, a saturated NaHCO₃ solution, and then brine. The organic phase was dried over anhydrous MgSO₄ and evaporated to dryness. The residue was purified to by silica gel column chromatography to produce Ethyl 2-O-(4-acetoxy-2,2-dimethylbutanoyl)-4-O-acetyl-3-O-chloroacetyl-6-O-[2,3,4,6-tetra-O-benzoyl)-β-D-glycopyranosyl]-1-thio-α-D-glycopyranoside 58(1.90 g, 78% for three steps). [H NMR δ 8.00-7.24 (m, 20H), 5.87 (t, J=9.6 Hz, 1H, H-3′), 5.65 (t, J=9.6 Hz, 1H, H-4′), 5.51 (dd, J=9.6 Hz and 8.0 Hz, 1H, H-2′), 5.47 (d, J=5.6 Hz, 1H, H-1), 5.38 (t, J=10.0 Hz, 1H, H-3), 4.87 (d, J=8.0 Hz, 1H, H-1′), 4.85 (t, J=10.0 Hz, 1H, H-4), 4.75 (dd, J=10.0 Hz and 6.0 Hz, 1H, H-2), 4.62 (dd, J=12.0 Hz and 2.8 Hz, 1H, H-6a′), 4.45 (dd, J=12.4 Hz and 5.2 Hz, 1H, H-6b′), 4.39 (m, 1H, H-5), 4.13 (m, 1H, H-5′), 4.01-3.99 (m, 3H), 3.89 (dd, J=18.4 Hz and 14.8 Hz, 2H), 3.60 (dd, J=11.2 Hz and 6.8 Hz, 1H, H-6b), 2.35-2.22 (m, 2H), 1.97 (s, 3H), 1.92 (s, 3H), 1.83-1.79 (m, 2H), 1.14 (s, 3H), 1.12 (s, 3H).1.02 (t, J=7.2 Hz, 3H).]

A mixture of Ethyl 2-O-(4-acetoxy-2,2-dimethylbutanoyl)-4-O-acetyl-3-O-chloroacetyl-6-O-[2,3,4,6-tetra-O-benzoyl)-β-D-glycopyranosyl]-1-thio-α-D-glycopyranoside 58 (0.8 g, 8.0 mmol), thiourea (304 mg, 5 eq.), and 2,6-dimethylpyridine (93 μL, 0.8 mmol) in MeOH (8 mL) and CH₂Cl₂ (12 mL) was boiled under reflux overnight. The mixture was concentrated and the residue was extracted with CH₂Cl₂. The extract was washed successively with cold dilute HCl, aqueous NaHCO₃, and H₂O. The organic layer was dried (MgSO₄), and concentrated. The residue was purified to by silica gel column chromatography to afford Ethyl 2-O-(4-acetoxy-2,2-dimethylbutanoyl)-4-O-acetyl-6-O-[2,3,4,6-tetra-O-benzoyl)-β-D-glycopyranosyl]-1-thio-α-D-glycopyranoside 60 (720 mg, 90%). [H NMR δ 8.00-7.24 (m, 20H), 5.86 (t, J=9.6 Hz, 1H, H-3′), 5.64 (t, J=9.6 Hz, 1H, H-4′), 5.51 (dd, J=9.6 Hz and 8.0 Hz, 1H, H-2′), 5.35 (d, J=6.0 Hz, 1H, H-1), 4.87 (d, J=8.0 Hz, 1H, H-1′), 4.75-4.62 (m, 3H), 4.47-3.90 (m, 7H), 3.63 (dd, J=10.8 Hz and 6.8 Hz, 1H, H-6b), 2.30-2.20 (m, 2H), 1.99 (s, 3H), 1.98 (s, 3H), 1.21 (s, 3H), 1.16 (s, 3H), 0.99 (t, J=7.2 Hz, 3H).]

To a stirred mixture of disaccharide 60 (818 mg, 0.82 mmol), imidate 62 (685 mg, 1.3 eq.), and molecular sieves (4 Å, 1.5 g) in CH₂Cl₂ (mL) at −70° C. was added TMSOTf (0.1 N in CH₂Cl₂, 0.8 mL). After stirring for 10 h, the reaction mixture was quenched with Et₃N (0.3 mL), filtered, and the filtrate was concentrated in vacuo. The residue was purified by silica gel column chromatography to give Ethyl 2-O-(4-acetoxy-2,2-dimethylbutanoyl)-3-O-[2-O-benzolyl-4,6-O-benzylidene-3-O-3-O-chloroacetyl-β-D-glycopyranosyl]-4-O-acetyl-6-O-[2,3,4,6-tetra-O-benzoyl)-β-D-glycopyranosyl]-1-thio-α-D-glycopyranoside 48 (1.07 g, 88%) as a white glass. [H NMR δ 7.94-7.19 (m, 25H), 5.80 (t, J=9.6 Hz, 1H, H-3″), 5.60 (t, J=9.6 Hz, 1H, H-4″), 5.44 (dd, J=9.6 Hz and 8.0 Hz, 1H, H-2′), 5.42 (s, 1H), 5.36 (d, J=5.6 Hz, 1H, H-1), 5.20 (t, J=9.2 Hz, 1H, H-2″), 4.82-4.74 (m, 4H, H-1, H-1′, H-2, H-3′), 4.65 (t, J=10.0 Hz, 1H), 4.57 (dd, J=12.4 Hz and 3.2 Hz, 1H), 4.42-4.29 (m, 3H), 4.11-3.90 (m, 9H), 3.68-3.44 (m, 4H), 2.20-2.09 (m, 2H), 1.92-1.83 (m, 2H), 1.96 (s, 3H), 1.93 (s, 3H), 1.89 (s, 3H), 1.75-1.69 (m, 2H), 1.21 (s, 3H), 1.18 (s, 3H), 1.05 (s, 6H), 0.89 (t, J=7.6 Hz, 3H). ¹³C NMR δ 175.9, 175.7, 171.2, 171.1, 169.6, 166.8, 166.3, 160.0, 165.4, 165.3, 136.6, 133.7, 133.6, 133.5, 133.4, 130.1, 130.0, 129.7, 129.6, 129.4, 128.9, 128.8, 128.7, 128.6, 128.6, 128.5, 128.5, 126.3, 101.9, 101.3, 100.1, 79.7, 78.5, 75.1, 74.1, 73.5, 73.1, 72.5, 72.3, 71.9, 69.7, 69.1, 68.7, 68.5, 66.6, 63.2, 61.5, 61.1, 41.4, 41.0, 40.8, 38.3, 38.1, 25.7, 25.6, 25.3, 24.9, 23.2, 21.2, 21.1, 21.0, 14.3.]

Although the disaccharide Ethyl 2-O-(4-acetoxy-2,2-dimethylbutanoyl)-3-O-chloroacetyl-6-O-[2,3,4,6-tetra-O-benzoyl)-β-D-glycopyranosyl]-1-thio-α-D-glycopyranoside 56 could be cleanly prepared without the ADMB group, numerous attempts to prepare the trisaccharide Ethyl 2-O-(4-acetoxy-2,2-dimethylbutanoyl)-3-O-[2-O-benzolyl-4,6-O-benzylidene-3-O-3-O-chloroacetyl-β-D-glycopyranosyl]-4-O-acetyl-6-O-[2,3,4,6-tetra-O-benzoyl)-β-D-glycopyranosyl]-1-thio-α-D-glycopyranoside 48 without resorting to the ADMB group were unsuccessful.

Synthesis of a linear decasaccharide (FIG. 2b) and a branched decasaccharide (FIG. 2a) are described here in a step-wise fashion ((a) through (h) for the unbranched molecule, and (a) through (h) for the branched molecule), referring to the appropriate figures to illustrate the reaction steps and chemical structures of reactants and products.

Synthesis of an Unbranched Decasaccharide

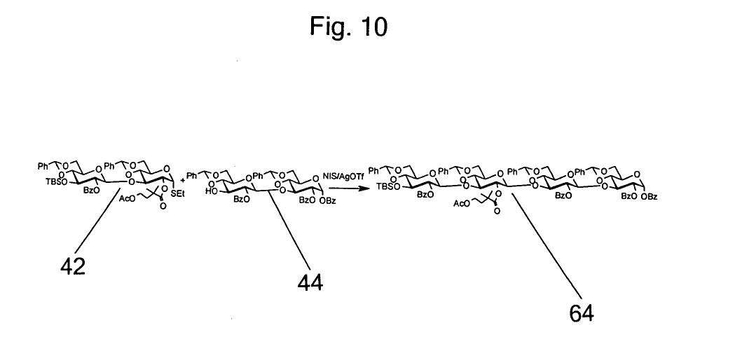

(a) FIG. 10

To a stirred mixture of Benzoyl 2-O-benzoyl-4,6-O-benzylidene-3-O-[2-O-benzoyl-4,6-O-benzylidene-β-D-glycopyranosyl]-α-D-glycopyranoside 44 (0.37 g, 0.44 mmol), thioglycoside S-Ethyl 2-O-(4-acetoxy-2,2-dimethylbutanoyl)-4,6-O-benzylidene-3-O-[2-O-benzoyl-4,6-O-benzylidene-3-O-(t-butyldimethylsilyl)]-1-thio-α-D-glycopyranoside 42 (0.5 g, 0.6 mmol), and molecular sieves (4 Å, 1 g) in dichloromethane (10 mL) at −20° C. were added N-iodosuccinimide (185 mg) and silver triflate (27 mg, in 1 mL dry toluene). After stirring for 3 h, the reaction mixture was quenched with Et₃N (0.1 mL), filtered, and the filtrate was concentrated in vacuo. The residue was purified by silica gel column chromatography to give Benzoyl 2-O-benzoyl-4,6-O-benzylidene-3-O-[2-O-benzoyl-4,6-O-benzylidene-3-O-[2-O-(4-acetoxy-2,2 -dimethylbutanoyl)-4,6-O-benzylidene-3-O-[2-O-benzoyl-4,6-O-benzylidene-3-O-(t-butyldimethylsilyl)-β-D-glycopyranosyl]-β-D-glycopyranosyl]-D-glycopyranosyl]-α-D-glycopyranoside 64 (698 mg, 92%). [H NMR δ 8.28-7.29 (m, 40H), 6.72 (d, J=4.0 Hz, 1H, H-1), 5.92 (s, 1H), 5.64 (s, 1H), 5.50 (dd, J=9.6 Hz and 4.0 Hz, 1H, H-2), 5.46 (t, J=8.8 Hz, 1H), 5.39 (s, 1H), 5.27 (d, J=8.0 Hz, 1H), 4.91 (d, J=7.6 Hz, 1H), 4.84-4.73 (m, 3H), 4.58 (t, J=9.6 Hz, 1H), 4.53-4.36 (m, 3H), 4.32-3.49 (m, 18H), 3.44 (t, J=10.0 Hz, 1H), 2.74 (t, J=9.6 Hz, 1H), 2.06 (s, 3H), 1.75-1.73 (m, 2H), 1.03 (s, 3H), 0.99 (s, 3H), 0.84 (s, 3H), 0.09 (s, 3H), 0.00 (s, 3H); ³C NMR δ 176.2, 175.8, 171.2, 165.6, 165.1, 164.0, 137.3, 134.0, 130.2, 130.0, 129.9, 129.4, 129.2, 129.1, 128.8, 128.6, 128.5, 128.3, 128.0, 126.8, 126.7, 126.5, 126.2, 102.3, 102.1, 102.0, 100.3, 99.3, 98.1, 96.3, 92.1, 82.2, 81.9, 79.5, 77.9, 76.1, 74.9, 74.8, 74.3, 73.7, 73.1, 72.9, 72.4, 68.9, 68.8, 68.6, 66.6, 66.2, 65.0, 63.1, 61.5, 61.2, 40.9, 40.7, 38.1, 29.9, 25.7, 25.3, 25.0, 21.2, 21.1, 18.1, 15.0, −4.1, −4.8.]

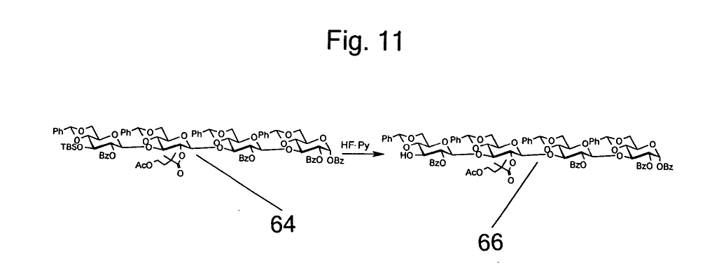

(b) FIG. 11

To a stirred solution of Benzoyl 2-O-benzoyl-4,6-O-benzylidene-3-O-[2-O-benzoyl-4,6-O-benzylidene-3-O-[2-O-(4-acetoxy-2,2-dimethylbutanoyl)-4,6-O-benzylidene-3-O-[2-O-benzoyl-4,6-O-benzylidene-3-O-(t-butyldimethylsilyl)-β-D-glycopyranosyl]-β-D-glycopyranosyl]-β-D-glycopyranosyl]-α-D-glycopyranoside 64 (700 mg, 0.41 mmol) in dry THF (10 mL) was added HF.Py (0.5 mL) at 0° C. under argon. After stirring for 24 h at room temperature, TLC showed that the starting material had disappeared. The mixture was diluted with ether, washed with aqueous NaHCO₃ and brine. The organic phase was dried over anhydrous MgSO₄ and evaporated to dryness. The crude product was subjected to flash column chromatography to produce Benzoyl 2-O-benzoyl-4,6-O-benzylidene-3-O-[2-O-benzoyl-4,6-O-benzylidene-3-O-[2-O-(4-acetoxy-2,2-dimethylbutanoyl)-4,6-O-benzylidene-3-O-[2-O-benzoyl-4,6-O-benzylidene-D-glycopyranosyl]-β-D-glycopyranosyl]-β-D-glycopyranosyl]-α-D-glycopyranoside 66 (587 mg, 90%). [¹H NMR (400 MHz, CDCl₃) δ 8.15-7.07 (m, 40H), 6.58 (d, J=3.6 Hz, 1H, H-1), 5.75 (s, 1H), 5.47 (s, 1H), 5.32 (dd, J=9.6 Hz and 4.0 Hz, 1H, H-2), 5.26 (t, J=8.4 Hz, 1H), 5.18 (s, 1H), 5.15 (d, J=7.6 Hz, 1H), 4.81 (d, J=7.6 Hz, 1H), 4.75-4.65 (m, 3H), 4.45 (t, J=9.2 Hz, 1H), 4.38-4.19 (m, 3H), 4.17-3.40 (m, 18H), 3.32 (t, J=9.6 Hz, 1H), 2.81 (t, J=9.6 Hz, 1H), 2.74 (bs, 1H), 1.90 (s, 3H), 1.62-1.58 (m, 2H), 0.90 (s, 3H), 0.86 (s, 3H).]

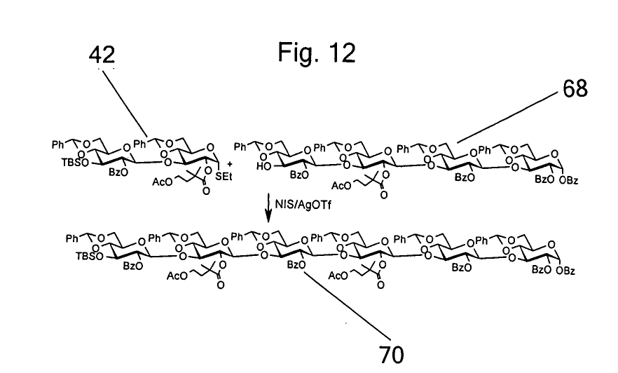

(c) FIG. 12

To a stirred mixture of tetrasaccharide Benzoyl 2-O-benzoyl-4,6-O-benzylidene-3-O-[2-O-benzoyl-4,6-O-benzylidene-3-O-[2-O-(4-acetoxy-2,2-dimethylbutanoyl)-4,6-O-benzylidene-3-O-[2-O-benzoyl-4,6-O-benzylidene-α-D-glycopyranosyl]-β-D-glycopyranosyl]-β-D-glycopyranosyl]-α-D-glycopyranoside 68 (370 mg, 0.23 mmol), thioglycoside S-Ethyl 2-O-(4-acetoxy-2,2-dimethylbutanoyl)-4,6-O-benzylidene-3-O-[2-O-benzoyl-4,6-O-benzylidene-3-O-(t-butyldimethylsilyl)]-1-thio-α-D-glycopyrano side 42 (262 mg, 1.2 eq.), and molecular sieves (4 Å, 800 mg) in CH₂Cl₂ (10 mL) at −20° C. were added NIS (97 mg) and AgOTf (15 mg, in 0.5 mL toluene). After stirring for 4 h, the reaction mixture was quenched with Et₃N (0.1 mL), filtered, and the filtrate was concentrated in vacuo, and the residue was purified by silica gel column chromatography to give Benzoyl 2-O-benzoyl-4,6-O-benzylidene-3-O-[2-O-benzoyl-4,6-O-benzylidene-3-O-[2-O-(4-acetoxy-2,2-dimethylbutanoyl)-4,6-O-benzylidene-3-O-[2-O-benzoyl-4,6-O-benzylidene-3-O-[2-O-(4-acetoxy-2,2-dimethylbutanoyl)-4,6-O-benzylidene-3-O-[2 -O-benzoyl-4,6-O-benzylidene-3-O-(t-butyldimethylsilyl)-β-D-glycopyranosyl]-β-D-glycopyranosyl]-β-D-glycopyranosyl]-β-D-glycopyranosyl]-β-D-glycopyranosyl]-α-D-glycopyranoside 70 (520 mg, 91%) as a white solid. [¹H NMR (400 MHz, CDCl₃) δ 8.33-7.09 (m, 55H), 6.67 (d, J=4.0 Hz, 1H), 5.84 (s, 1H), 5.63 (s, 1H), 5.46-5.30 (m, 5H), 5.02 (d, d, J=8.0 Hz, 1H), 4.98-4.84 (m, 4H), 4.75-4.38 (m, 8H), 4.36-3.41 (m, 34H), 2.69-2.43 (m, 2H), 2.09 (s, 3H), 2.03 (s, 3H), 1.70-1.66 (m, 4H), 1.06 (s, 3H), 1.02 (s, 3H), 0.99 (s, 3H), 0.94 (s, 3H), 0.83 (s, 9H), 0.08(s, 3H), 0.00 (s, 3H); ¹³C NMR δ 175.95, 175.84, 171.10, 170.95, 166.14, 165.30, 164.92, 164.62, 164.59 (9×C(═O)), 102.90, 102.63, 102.09, 101.78, 101.02, 100.40, 100.37, 96.68, 96.32, 96.22, 96.16, 90.49 (6×PhCH and 6×C₁).]

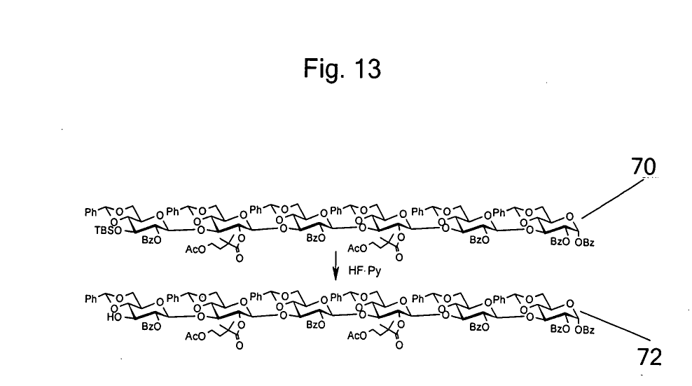

(d) FIG. 13

As shown in FIG. 13, to a solution of Benzoyl 2-O-benzoyl-4,6-O-benzylidene-3-0-[2-O-benzoyl-4,6-O-benzylidene-3-O-[2-O-(4-acetoxy-2,2-dimethylbutanoyl)-4,6-O-benzylidene-3-O-[2-O-benzoyl-4,6-O-benzylidene-3-O-[2-O-(4-acetoxy-2,2-dimethylbutanoyl)-4,6-O-benzylidene-3-O-[2-O-benzoyl-4,6-O-benzylidene-3-O-(t-butyldimethylsilyl)-β-D-glycopyranosyl]-β-D-glycopyranosyl]-β-D-glycopyranosyl]-β-D-glycopyranosyl]-β-D-glycopyranosyl]-α-D-glycopyranoside 70 in dry THF (10 mL) was added HF.Py (0.5 mL) at 0° C. under argon. After stirring for 24 h at room temperature, TLC showed that the starting material had disappeared. The mixture was diluted with ether, washed with aqueous NaHCO₃ and brine. The organic phase was dried over anhydrous MgSO₄ and evaporated to dryness. The crude product was subjected to flash column chromatography to produce Benzoyl 2-O-benzoyl-4,6-O-benzylidene-3-O-[2-O-benzoyl-4,6-O-benzylidene-3-O-[2-O-(4-acetoxy-2,2-dimethylbutanoyl)-4,6-0 -benzylidene-3-O-[2-O-benzoyl-4,6-O-benzylidene-3-O-[2-O-(4-acetoxy-2,2-dimethylbutanoyl)-4,6-O-benzylidene-3-O-[2-O-benzoyl-4,6-O-benzylidene-β-D-glycopyranosyl]-β-D-glycopyranosyl]-D-glycopyranosyl]-β-D-glycopyranosyl]-β-D-glycopyranosyl]-α-D-glycopyranoside 72. [H NMR δ 8.40-7.26 (m, 55H), 6.74 (d, J=4.0 Hz, 1H), 5.92 (s, 1H), 5.70 (s, 1H), 5.55-5.37 (m, 5H), 5.17-5.11 (m, 2H), 5.09-4.71 (m, 6H), 4.61-4.38 (m, 4H), 4.38-3.87 (m, 20H), 3.82-3.48 (m, 10H), 2.78-2.68 (m, 2H), 2.09 (s, 3H), 2.05 (s, 3H), 1.93-1.71 (m, 4H), 1.13 (s, 3H), 1.10 (s, 3H), 1.06 (s, 3H), 1.01 (s, 3H); ³C NMR δ 176.0, 175.9, 171.1, 171.0, 166.2, 165.3, 164.9, 164.6, 164.6, 137.6, 137.5, 137.3, 134.8, 134.5, 134.1, 133.6, 133.2, 130.3, 130.1, 130.0, 129.7, 129.6, 129.4, 129.3, 129.1, 128.9, 128.7, 128.6, 128.1, 126.9, 126.6, 126.4, 102.9, 102.6, 102.1, 101.8, 101.0, 100.4, 96.7, 96.3, 96.2, 90.5, 81.3, 79.4, 78.5, 78.2, 75.8, 75.1, 74.6, 74.5, 72.7, 72.6, 72.2, 71.5, 71.4, 69.0, 68.8, 68.6, 66.4, 66.2, 65.5, 64.8, 64.6, 61.1, 61.0, 40.7, 40.5, 38.1, 38.0,25.0, 24.8, 21.2, 21.1.]

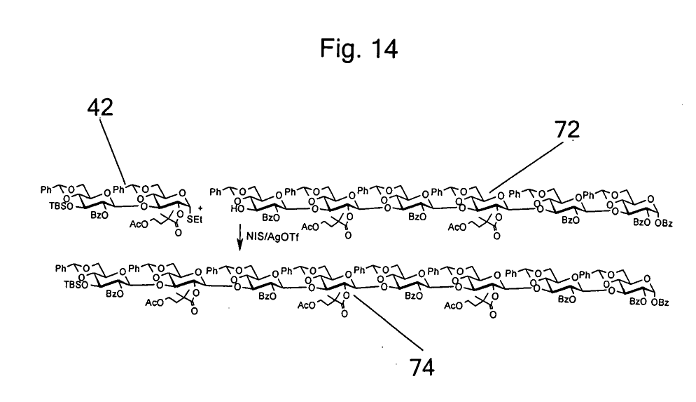

(e) FIG. 14

To a stirred mixture of hexasaccharide Benzoyl 2-O-benzoyl-4,6-O-benzylidene-3-O-[2-O-benzoyl-4,6-O-benzylidene-3-O-[2-O-(4-acetoxy-2,2-dimethylbutanoyl)-4,6-O-benzylidene-3-O-[2-O-benzoyl-4,6-O-benzylidene-3-O-[2-O-(4-acetoxy-2,2-dimethylbutanoyl)-4,6-O-benzylidene-3-O-[2-O-benzoyl-4,6-O-benzylidene-β-D-glycopyranosyl]-β-D-glycopyranosyl]-β-D-glycopyranosyl]-β-D-glycopyranosyl]-β-D-glycopyranosyl]-α-D-glycopyranoside 72 (285 mg, 0.12 mmol), thioglycoside S-Ethyl 2-O-(4-acetoxy-2,2-dimethylbutanoyl)-4,6-O-benzylidene-3-O-[2-O-benzoyl-4,6-O-benzylidene-3-O-(t-butyldimethylsilyl)]-1-thio-α-D-glycopyranoside 42 (170 mg, 1.5 eq.), and molecular sieves (4 Å, 400 mg) in CH₂Cl₂ (5 mL) at −20° C. were added NIS (50 mg) and AgOTf (6 mg, in 0.5 mL dry toluene). After stirring for 4 h, the reaction mixture was quenched with Et₃N (0.1 mL), filtered, and the filtrate was concentrated in vacuo. The residue was purified by silica gel column chromatography to give Benzoyl 2-O-benzoyl-4,6-O-benzylidene-3-O-[2-O-benzoyl-4,6-O-benzylidene-3-O-[2-O-(4-acetoxy-2,2-dimethylbutanoyl)-4,6-O-benzylidene-3-O-[2-O-benzoyl-4,6-O-benzylidene-3-O-[2-O-(4-acetoxy-2,2-dimethylbutanoyl)-4,6-O-benzylidene-3-O-[2-O-benzoyl-4,6-O-benzylidene-3-O-[2-O-(4-acetoxy-2,2-dimethylbutanoyl)-4,6-O-benzylidene-3-O-[2-O-benzoyl-4,6-O-benzylidene-3-O-(t-butyldimethylsilyl)-β-D-glycopyranosyl]-β-D-glycopyranosyl]-β-D-glycopyranosyl]-β-D-glycopyranosyl]-β-D-glycopyranosyl]-β-D-glycopyranosyl]-β-D-glycopyranosyl]-α-D-glycopyranoside 74 (351 mg, 90%) as a white solid. [H NMR δ 8.38-7.10 (m, 70H), 6.67 (d, J=4.0 Hz, 1H), 5.84 (s, 1H), 5.64 (s, 1H), 5.49-5.33 (m, 6H), 5.11 (d, J=8.4 Hz, 1H), 5.04 (d, J=8.8 Hz, 1H), 4.92-4.62 (m, 10H), 4.54-4.36 (m, 4H), 4.30-3.40 (m, 45H), 2.65-2.42 (m, 2H), 2.09 (s, 3H), 2.08 (s, 3H), 2.03 (s, 3H), 1.86-1.64 (m, 6H), 1.07 (s, 3H), 1.06 (s, 3H), 1.03 (s, 3H), 1.02 (s, 3H), 0.98 (s, 3H), 0.94 (s, 3H), 0.83 (s, 9H), 0.08 (s, 3H), 0.01 (s, 3H); ¹³C NMR δ 180.7, 180.6, 180.5, 175.7, 175.6, 175.5, 170.2, 169.9, 169.8, 169.6, 169.3, 169.2, 142.4, 142.3, 142.2, 142.1, 142.0, 141.9, 139.4, 139.2, 138.7, 138.2, 137.9, 135.1, 134.7, 134.5, 134.3, 134.2, 134.1, 134.0, 133.8, 133.7, 133.6, 133.5, 133.3, 133.2, 133.1, 132.9, 132.7, 132.6, 132.5, 131.6, 131.5, 131.1, 131.0, 130.9, 130.9, 107.5, 107.3, 106.5, 106.4, 105.7, 105.0, 104.9, 104.8, 101.3, 100.9, 100.7, 100.6, 95.1, 86.5, 84.0, 83.2, 83.0, 82.8, 82.2, 81.5, 80.5, 79.7, 79.3, 79.2, 79.1, 77.9, 76.9, 76.8, 76.7, 76.1, 75.8, 73.5, 73.2, 71.3, 71.1, 70.9, 70.8, 70.1, 69.4, 65.7, 65.6, 45.3, 45.3, 45.2, 42.8, 42.6, 30.4, 29.7, 29.6, 29.5, 29.4, 25.8, 25.7, 22.8, 0.58, 0.00.]

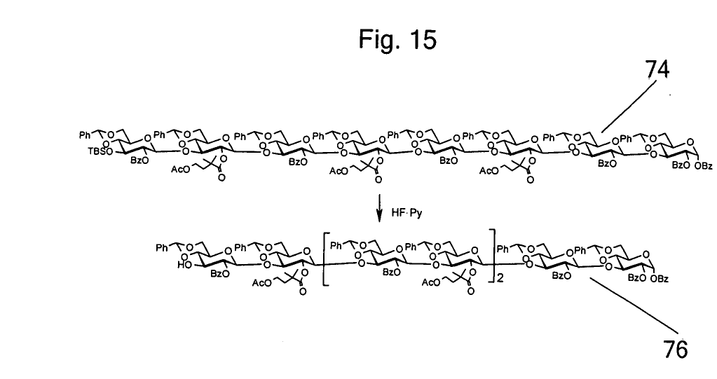

(f) FIG. 15

To a stirred solution of compound Benzoyl 2-O-benzoyl-4,6-O-benzylidene-3-O-[2-O-benzoyl-4,6-O-benzylidene-3-O-[2-O-(4-acetoxy-2,2-dimethylbutanoyl)-4,6-O-benzylidene-3-O-[2-O-benzoyl-4,6-O-benzylidene-3-O-[2-O-(4-acetoxy-2,2-dimethylbutanoyl)-4,6-O-benzylidene-3-O-[2-O-benzoyl-4,6-O-benzylidene-3-O-[2-O-(4-acetoxy-2,2-dimethylbutanoyl)-4,6-O-benzylidene-3-O-[2-O-benzoyl-4,6-O-benzylidene-3-O-(t-butyldimethylsilyl)-β-D-glycopyranosyl]-β-D-glycopyranosyl]-β-D-glycopyranosyl]-β-D-glycopyranosyl]-β3-D-glycopyranosyl]-β-D-glycopyranosyl]-β-D-glycopyranosyl]-α-D-glycopyranoside 74 (300 mg, 0.093 mmol) in dry THF (5 mL) was added HF.Py (0.5 mL) at 0° C. under Argon. After stirring for 24 h at room temperature, TLC showed that the starting material disappeared. The mixture was diluted with ether, washed with aqueous NaHCO₃ and brine. The organic phase was dried over anhydrous MgSO₄ and evaporated to dryness. The crude product was subjected to flash column chromatography to afford Benzoyl 2-O-benzoyl-4,6-O-benzylidene-3-O-[2-O-benzoyl-4,6-O-benzylidene-3-O-[2-O-(4-acetoxy-2,2-dimethylbutanoyl)-4,6-O-benzylidene-3-O-[2-O-benzoyl-4,6-O-benzylidene-3-O-[2-O-(4-acetoxy-2,2-dimethylbutanoyl)-4,6-O-benzylidene-3-O-[2-O-benzoyl-4,6-O-benzylidene-3-O-[2-O-(4-acetoxy-2,2-dimethylbutanoyl)-4,6-O-benzylidene-3-O-[2-O-benzoyl-4,6-O-benzylidene-α-D-glycopyranosyl]-β-D-glycopyranosyl]-β-D-glycopyranosyl]-β-D-glycopyranosyl]-β-D-glycopyranosyl]-β-D-glycopyranosyl]-β-D-glycopyranosyl]-α-D-glycopyranoside 76 (260 mg, 90%). [¹H NMR (400 MHz, CDCl₃) δ 8.28-7.10 (m, 70H), 6.67 (d, J=4.0 Hz, 1H), 5.84 (s, 1H), 5.64 (s, 1H), 5.49-5.33 (m, 6H), 5.11 (d, J=8.4 Hz, 1H), 5.04 (d, J=8.8 Hz, 1H), 4.92-4.62 (m, 10H), 4.54-4.36 (m, 4H), 4.30-3.40 (m, 45H), 2.65-2.42 (m, 2H), 2.09 (s, 3H), 2.08 (s, 3H), 2.03 (s, 3H), 1.86-1.64 (m, 6H), 1.07 (s, 3H), 1.06 (s, 3H), 1.03 (s, 3H), 1.02 (s, 3H), 0.98 (s, 3H), 0.94 (s, 3H).]

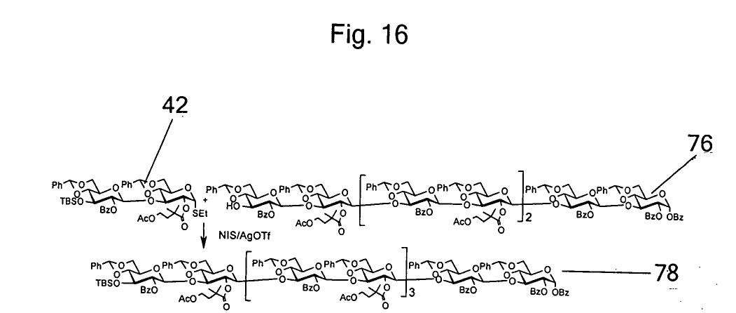

(g) FIG. 16

To a stirred mixture of octasaccharide Benzoyl 2-O-benzoyl-4,6-O-benzylidene-3-O-[2-O-benzoyl-4,6-O-benzylidene-3-O-[2-O-(4-acetoxy-2,2-dimethylbutanoyl)-4,6-O-benzylidene-3-O-[2-O-benzoyl-4,6-O-benzylidene-3-O-[2-O-(4-acetoxy-2,2-dimethylbutanoyl)-4,6-O-benzylidene-3-O-[2-O-benzoyl-4,6-O-benzylidene-3-O-[2-O-(4-acetoxy-2,2-dimethylbutanoyl)-4,6-O-benzylidene-3-O-[2-O-benzoyl-4,6-O-benzylidene-β-D-glycopyranosyl]-β-D-glycopyranosyl]-β-D-glycopyranosyl]-β-D-glycopyranosyl]-β-D-glycopyranosyl]-β-D-glycopyranosyl]-β-D-glycopyranosyl]-α-D-glycopyranoside 76 (170 mg, 0.054 mmol), thioglycoside S-Ethyl 2-O-(4-acetoxy-2,2-dimethylbutanoyl)-4,6-O-benzylidene-3-O-[2-O-benzoyl-4,6-O-benzylidene-3-O-(t-butyldimethylsilyl)]-1-thio-α-D-glycopyranoside 42 (100 mg, 2 eq.), and molecular sieves (4 Å, 100 mg) in CH₂Cl₂ (5 mL) at −20° C. were added NIS (27 mg) and AgOTf (3 mg in 0.3 mL dry toluene). After stirring for 10 h, the reaction mixture was quenched with Et₃N (0.1 mL), filtered, and the filtrate was concentrated in vacuo. The residue was purified by silica gel column chromatography to give Benzoyl 2-O-benzoyl-4,6-O-benzylidene-3-O-[2-O-benzoyl-4,6-O-benzylidene-3-O-[2-O-(4-acetoxy-2,2-dimethylbutanoyl)-4,6-O-benzylidene-3-O-[2-O-benzoyl-4,6-O-benzylidene-3-O-[2-O-(4-acetoxy-2,2-dimethylbutanoyl)-4,6-O-benzylidene-3-O-[2-O-benzoyl-4,6-O-benzylidene-3-O-[2-O-(4-acetoxy-2,2-dimethylbutanoyl)-4,6-O-benzylidene-3-O-[2-O-benzoyl-4,6-O-benzylidene-3-O-[2-O-(4-acetoxy-2,2-dimethylbutanoyl)-4,6-O-benzylidene-3-O-[2-O-benzoyl-4,6-O-benzylidene-3-O-(t-butyldimethylsilyl)-β-D-glycopyranosyl]-β-D-glycopyranosyl]-β-D-glycopyranosyl]-α-D-glycopyranosyl]-β-D-glycopyranosyl]-D-glycopyranosyl]-β-D-glycopyranosyl]-β-D-glycopyranosyl]-β-D-glycopyranosyl]-α-D-glycopyranoside 78 (195 mg, 90%). [H NMR δ 8.45-7.18 (m, 105H), 6.74 (d, J=4.0 Hz, 1H), 5.92 (s, 1H), 5.71 (s, 1H), 5.56-5.34 (m, 8H), 5.22-5.18 (m, 2H), 5.12 (d, J=8.4 Hz, 1H), 4.99-4.69 (m, 13H), 4.61-4.45 (m, 5H), 4.39-3.92 (m, 36H), 3.89-3.47 (m, 20H), 2.91-2.50 (m, 4H), 2.18 (s, 3H), 2.17 (s, 3H), 2.16 (s, 3H), 2.11 (s, 3H), 1.93-1.73 (m, 8H), 1.15 (s, 3H), 1.14 (s, 3H), 1.14 (s, 3H), 1.11 (s, 3H), 1.10 (s, 3H), 1.10 (s, 3H), 1.06 (s, 3H), 1.01 (s, 3H), 0.99 (s, 9H).]

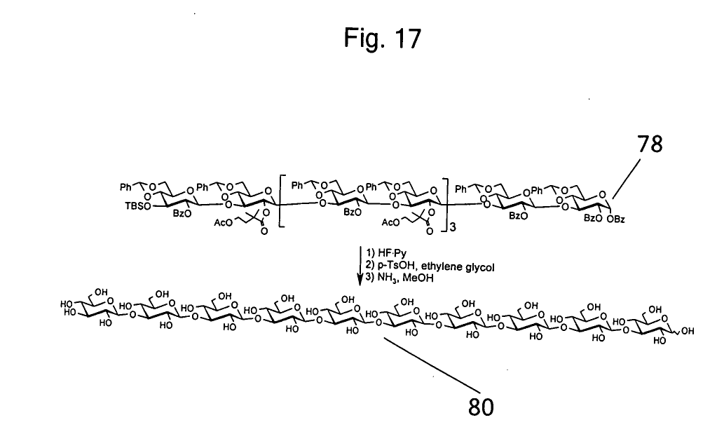

(h) FIG. 17

To a stirred solution of compound Benzoyl 2-O-benzoyl-4,6-O-benzylidene-3-O-[2-O-benzoyl-4,6-O-benzylidene-3-O-[2-O-(4-acetoxy-2,2-dimethylbutanoyl)-4,6-O-benzylidene-3-O-[2-O-benzoyl-4,6-O-benzylidene-3-O-[2-O-(4-acetoxy-2,2-dimethylbutanoyl)-4,6-O-benzylidene-3-O-[2-O-benzoyl-4,6-O-benzylidene-3-O-[2-O-(4-acetoxy-2,2-dimethylbutanoyl)-4,6-O-benzylidene-3-O-[2-O-benzoyl-4,6-O-benzylidene-3-O-[2-O-(4-acetoxy-2,2-dimethylbutanoyl)-4,6-O-benzylidene-3-O-[2-O-benzoyl-4,6-O-benzylidene-3-O-(t-butyldimethylsilyl)-β-D-glycopyranosyl]-β-D-glycopyranosyl]-β-D-glycopyranosyl]-β-D-glycopyranosyl]-β-D-glycopyranosyl]-β-D-glycopyranosyl]-β-D-glycopyranosyl]-β-D-glycopyranosyl]-β-D-glycopyranosyl]-α-D-glycopyranoside 78 (0.16 g, 0.040 mmol) in dry THF (5 mL) was added HF.Py (0.4 mL) at 0° C. under argon. After stirring for 24 h at room temperature, TLC showed that the reaction was complete. The mixture was diluted with ether, washed with aqueous NaHCO₃ and brine. The organic phase was dried over anhydrous MgSO₄ and evaporated to dryness. To the residue and ethylene glycol (0.2 mL) in CH₃CN (4 mL) was added p-toluenesulfonic acid (5 mg). The mixture was stirred overnight at room temperature, then neutralized with Et₃N and concentrated. After a short flash chromatography, the fractions were collected and concentrated. The residue was dissolved in 10 mL MeOH/NH₃, and stirred for overnight. After concentration, the residue was purified by Bio-gel P2 using water as the eluant. The fractions were collected and lyophilized to give the amorphous unbranched decasaccharide 80 (58 mg, 89%). [¹H NMR (400 MHz, D₂O) 84.57 (d, J=8.0 Hz, 0.7H), 4.53 (d, J=8.0 Hz, 0.3H), 3.70 (bd, J=11.6 Hz, 1H), 3.59-3.49 (m, 2H), 3.33 (t, J=8.0 Hz, 1H), 3.30-3.25 (m, 2H).]

Synthesis of a Branched Decasaccharide

(a) FIG. 10

To a stirred mixture of Benzoyl 2-O-benzoyl-4,6-O-benzylidene-3-O-[2-O-benzoyl-4,6-O-benzylidene-β-D-glycopyranosyl]-α-D-glycopyranoside 44 (0.37 g, 0.44 mmol), thioglycoside S-Ethyl 2-O-(4-acetoxy-2,2-dimethylbutanoyl)-4,6-O-benzylidene-3-O-[2-O-benzoyl-4,6-O-benzylidene-3-O-(t-butyldimethylsilyl)]-1-thio-α-D-glycopyranoside 42 (0.5 g, 0.6 mmol), and molecular sieves (4 Å, 1 g) in dichloromethane (10 mL) at −20° C. were added N-iodosuccinimide (185 mg) and silver triflate (27 mg, in 1 mL dry toluene). After stirring for 3 h, the reaction mixture was quenched with Et₃N (0.1 mL), filtered, and the filtrate was concentrated in vacuo. The residue was purified by silica gel column chromatography to give Benzoyl 2-O-benzoyl-4,6-O-benzylidene-3-O-[2-O-benzoyl-4,6-O-benzylidene-3-O-[2-O-(4-acetoxy-2,2-dimethylbutanoyl)-4,6-O-benzylidene-3-O-[2-O-benzoyl-4,6-O-benzylidene-3-O-(t-butyldimethylsilyl)-β-D-glycopyranosyl]-α-D-glycopyranosyl]-β-D-glycopyranosyl]-α-D-glycopyranoside 64 (698 mg, 92%). [H NMR δ 8.28-7.29 (m, 40H), 6.72 (d, J=4.0 Hz, 1H, H-1), 5.92(s, 1H), 5.64(s, 1H), 5.50(dd, J=9.6 Hz and 4.0 Hz, 1H, H-2), 5.46(t, J=8.8 Hz, 1H), 5.39 (s, 1H), 5.27 (d, J=8.0 Hz, 1H), 4.91 (d, J=7.6 Hz, 1H), 4.84-4.73 (m, 3H), 4.58 (t, J=9.6 Hz, 1H), 4.53-4.36 (m, 3H), 4.32-3.49 (m, 18H), 3.44 (t, J=10.0 Hz, 1H), 2.74 (t, J=9.6 Hz, 1H), 2.06 (s, 3H), 1.75-1.73 (m, 2H), 1.03 (s, 3H), 0.99 (s, 3H), 0.84 (s, 3H), 0.09 (s, 3H), 0.00 (s, 3H); ³C NMR δ 176.2, 175.8, 171.2, 165.6, 165.1, 164.0, 137.3, 134.0, 130.2, 130.0, 129.9, 129.4, 129.2, 129.1, 128.8, 128.6, 128.5, 128.3, 128.0, 126.8, 126.7, 126.5, 126.2, 102.3, 102.1, 102.0, 100.3, 99.3, 98.1, 96.3, 92.1, 82.2, 81.9, 79.5, 77.9, 76.1, 74.9, 74.8, 74.3, 73.7, 73.1, 72.9, 72.4, 68.9, 68.8, 68.6, 66.6, 66.2, 65.0, 63.1, 61.5, 61.2, 40.9, 40.7, 38.1, 29.9, 25.7, 25.3, 25.0, 21.2, 21.1, 18.1, 15.0, −4.1, −4.8.]

(b) FIG. 11

To a stirred solution of compound Benzoyl 2-O-benzoyl-4,6-O-benzylidene-3-O-[2-O-benzoyl-4,6-O-benzylidene-3-O-[2-O-(4-acetoxy-2,2-dimethylbutanoyl)-4,6-O-benzylidene-3-O-[2-O-benzoyl-4,6-O-benzylidene-3-O-(t-butyldimethylsilyl)-β-D-glycopyranosyl]-β-D-glycopyranosyl]-β-D-glycopyranosyl]-α-D-glycopyranoside 64 (700 mg, 0.41 mmol) in dry THF (10 mL) was added HF.Py (0.5 mL) at 0° C. under argon. After stirring for 24 h at room temperature, TLC showed that the starting material had disappeared. The mixture was diluted with ether, washed with aqueous NaHCO₃ and brine. The organic phase was dried over anhydrous MgSO₄ and evaporated to dryness. The crude product was subjected to flash column chromatography to produce Benzoyl 2-O-benzoyl-4,6-O-benzylidene-3-O-[2-O-benzoyl-4,6-O-benzylidene-3-O-[2-O-(4-acetoxy-2,2-dimethylbutanoyl)-4,6-O-benzylidene-3-O-[2-O-benzoyl-4,6-O-benzylidene-β-D-glycopyranosyl]-β-D-glycopyranosyl]-β-D-glycopyranosyl]-α-D-glycopyranoside 66 (587 mg, 90%). [¹H NMR (400 MHz, CDCl₃) δ 8.15-7.07 (m, 40H), 6.58 (d, J=3.6 Hz, 1H, H-1), 5.75 (s, 1H), 5.47 (s, 1H), 5.32 (dd, J=9.6 Hz and 4.0 Hz, 1H, H-2), 5.26 (t, J=8.4 Hz, 1H), 5.18 (s, 1H), 5.15 (d, J=7.6 Hz, 1H), 4.81 (d, J=7.6 Hz, 1H), 4.75-4.65 (m, 3H), 4.45 (t, J=9.2 Hz, 1H), 4.38-4.19 (m, 3H), 4.17-3.40 (m, 18H), 3.32 (t, J=9.6 Hz, 1H), 2.81 (t, J=9.6 Hz, 1H), 2.74 (bs, 1H), 1.90 (s, 3H), 1.62-1.58 (m, 2H), 0.90 (s, 3H), 0.86 (s, 3H).]

(c) FIG. 12

To a stirred mixture of tetrasaccharide Benzoyl 2-O-benzoyl-4,6-O-benzylidene-3-O-[2-O-benzoyl-4,6-O-benzylidene-3-O-[2-O-(4-acetoxy-2,2-dimethylbutanoyl)-4,6-O-benzylidene-3-O-[2-O-benzoyl-4,6-O-benzylidene-β-D-glycopyranosyl]-β-D-glycopyranosyl]-β-D-glycopyranosyl]-α-D-glycopyranoside 68 (370 mg, 0.23 mmol), thioglycoside S-Ethyl 2-O-(4-acetoxy-2,2-dimethylbutanoyl)-4,6-O-benzylidene-3-O-[2-O-benzoyl-4,6-O-benzylidene-3-O-(t-butyldimethylsilyl)]-1-thio-α-D-glycopyranoside 42 (262 mg, 1.2 eq.), and molecular sieves (4 Å, 800 mg) in CH₂Cl₂ (10 mL) at −20° C. were added NIS (97 mg) and AgOTf (15 mg, in 0.5 mL toluene). After stirring for 4 h, the reaction mixture was quenched with Et₃N (0.1 mL), filtered, and the filtrate was concentrated in vacuo, and the residue was purified by silica gel column chromatography to give Benzoyl 2-O-benzoyl-4,6-O-benzylidene-3-O-[2-O-benzoyl-4,6-O-benzylidene-3-O-[2-O-(4-acetoxy-2,2-dimethylbutanoyl)-4,6-O-benzylidene-3-O-[2-O-benzoyl-4,6-O-benzylidene-3-O-[2-O-(4-acetoxy-2,2-dimethylbutanoyl)-4,6-O-benzylidene-3-O-[2-O-benzoyl-4,6-O-benzylidene-3-O-(t-butyldimethylsilyl)-β-D-glycopyranosyl]-β-D-glycopyranosyl]-β-D-glycopyranosyl]-β-D-glycopyranosyl]-β-D-glycopyranosyl]-α-D-glycopyranoside 70 (520 mg, 91%) as a white solid. [¹H NMR (400 MHz, CDCl₃) δ 8.33-7.09 (m, 55H), 6.67 (d, J=4.0 Hz, 1H), 5.84 (s, 1H), 5.63 (s, 1H), 5.46-5.30 (m, 5H), 5.02 (d, d, J=8.0 Hz, 1H), 4.98-4.84 (m, 4H), 4.75-4.38 (m, 8H), 4.36-3.41 (m, 34H), 2.69-2.43 (m, 2H), 2.09 (s, 3H), 2.03 (s, 3H), 1.70-1.66 (m, 4H), 1.06 (s, 3H), 1.02 (s, 3H), 0.99 (s, 3H), 0.94 (s, 3H), 0.83 (s, 9H), 0.08(s, 3H), 0.00 (s, 3H); ¹³C NMR δ 175.95, 175.84, 171.10, 170.95, 166.14, 165.30, 164.92, 164.62, 164.59 (9×C(═O)), 102.90, 102.63, 102.09, 101.78, 101.02, 100.40, 100.37, 96.68, 96.32, 96.22, 96.16, 90.49 (6×PhCH and 6×C₁).]

(d) FIG. 13

To a solution of Benzoyl 2-O-benzoyl-4,6-O-benzylidene-3-O-[2-O-benzoyl-4,6-O-benzylidene-3-O-[2-O-(4-acetoxy-2,2-dimethylbutanoyl)-4,6-O-benzylidene-3-O-[2-O-benzoyl-4,6-O-benzylidene-3-O-[2-O-(4-acetoxy-2,2-dimethylbutanoyl)-4,6-O-benzylidene-3-O-[2-O-benzoyl-4,6-O-benzylidene-3-O-(t-butyldimethylsilyl)-β-D-glycopyranosyl]-β-D-glycopyranosyl]-β-D-glycopyranosyl]-β-D-glycopyranosyl]-β-D-glycopyranosyl]-α-D-glycopyranoside 70 in dry THF (10 mL) was added HF.Py (0.5 mL) at 0° C. under argon. After stirring for 24 h at room temperature, TLC showed that the starting material had disappeared. The mixture was diluted with ether, washed with aqueous NaHCO₃ and brine. The organic phase was dried over anhydrous MgSO₄ and evaporated to dryness. The crude product was subjected to flash column chromatography to produce Benzoyl 2-O-benzoyl-4,6-O-benzylidene-3-O-[2-O-benzoyl-4,6-O-benzylidene-3-O-[2-O-(4-acetoxy-2,2-dimethylbutanoyl)-4,6-O-benzylidene-3-O-[2-O-benzoyl-4,6-O-benzylidene-3-O-[2-O-(4-acetoxy-2,2-dimethylbutanoyl)-4,6-O-benzylidene-3-O-[2-O-benzoyl-4,6-O-benzylidene-β-D-glycopyranosyl]-β-D-glycopyranosyl]-β-D-glycopyranosyl]-β-D-glycopyranosyl]-β-D-glycopyranosyl]-α-D-glycopyranoside 72 [H NMR δ 8.40-7.26 (m, 55H), 6.74 (d, J=4.0 Hz, 1H), 5.92 (s, 1H), 5.70 (s, 1H), 5.55-5.37 (m, 5H), 5.17-5.11 (m, 2H), 5.09-4.71 (m, 6H), 4.61-4.38 (m, 4H), 4.38-3.87 (m, 20H), 3.82-3.48 (m, 10H), 2.78-2.68 (m, 2H), 2.09 (s, 3H), 2.05 (s, 3H), 1.93-1.71 (m, 4H), 1.13 (s, 3H), 1.10 (s, 3H), 1.06 (s, 3H), 1.01 (s, 3H); ¹³C NMR δ 176.0, 175.9, 171.1, 171.0, 166.2, 165.3, 164.9, 164.6, 164.6, 137.6, 137.5, 137.3, 134.8, 134.5, 134.1, 133.6, 133.2, 130.3, 130.1, 130.0, 129.7, 129.6, 129.4, 129.3, 129.1, 128.9, 128.7, 128.6, 128.1, 126.9, 126.6, 126.4, 102.9, 102.6, 102.1, 101.8, 101.0, 100.4, 96.7, 96.3, 96.2, 90.5, 81.3, 79.4, 78.5, 78.2, 75.8, 75.1, 74.6, 74.5, 72.7, 72.6, 72.2, 71.5, 71.4, 69.0, 68.8, 68.6, 66.4, 66.2, 65.5, 64.8, 64.6, 61.1, 61.0, 40.7, 40.5, 38.1, 38.0, 25.0, 24.8, 21.2, 21.1.]

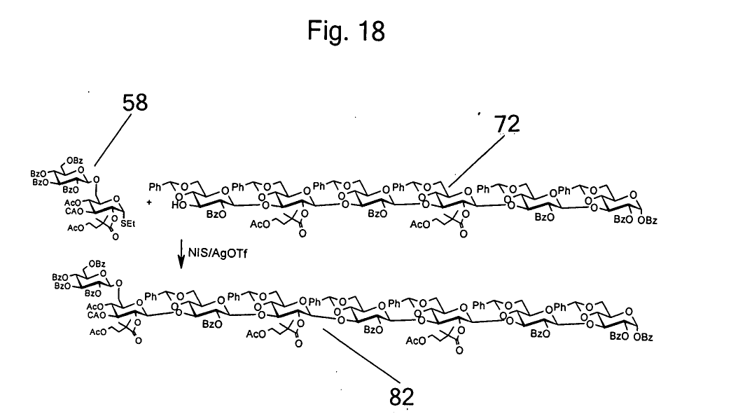

(e) FIG. 18

To a stirred mixture of hexasaccharide Benzoyl 2-O-benzoyl-4,6-O-benzylidene-3-O-[2-O-benzoyl-4,6-O-benzylidene-3-O-[2-O-(4-acetoxy-2,2-dimethylbutanoyl)-4,6-O-benzylidene-3-O-[2-O-benzoyl-4,6-O-benzylidene-3-O-[2-O-(4-acetoxy-2,2-dimethylbutanoyl)-4,6-O-benzylidene-3-O-[2-O-benzoyl-4,6-O-benzylidene-β-D-glycopyranosyl]-β-D-glycopyranosyl]-β-D-glycopyranosyl]-β-D-glycopyranosyl]-β-D-glycopyranosyl]-α-D-glycopyranoside 72 (300 mg, 0.127 mmol), thioglycoside Ethyl 2-O-(4-acetoxy-2,2-dimethylbutanoyl)-4-O-acetyl-3-O-chloroacetyl-6-O-[2,3,4,6-tetra-O-benzoyl)-β-D-glycopyranosyl]-1-thio-α-D-glycopyranoside 58 (250 mg, 1.9 eq.), and molecular sieves (4 Å, 100 mg) in CH₂Cl₂ (10 mL) at −20° C. were added NIS (71 mg) and AgOTf (7 mg in 0.5 mL dry toluene). After stirring for 10 h, the reaction mixture was quenched with Et₃N (0.1 mL), filtered, and the filtrate was concentrated in vacuo. The residue was purified by silica gel column chromatography to give Benzoyl 2-O-benzoyl-4,6-O-benzylidene-3-O-[2-O-benzoyl-4,6-O-benzylidene-3-O-[2-O-(4-acetoxy-2,2-dimethylbutanoyl)-4,6-O-benzylidene-3-O-[2-O-benzoyl-4,6-O-benzylidene-3-O-[2-O-(4-acetoxy-2,2-dimethylbutanoyl)-4,6-O-benzylidene-3-O-[2-O-benzoyl-4,6-O-benzylidene-3-O-[2-O-(4-acetoxy-2,2-dimethylbutanoyl)-4-O-Acetyl-3-O-chloroacetyl-6-O-[2,3,4,6-tetra-O-benzoyl-α-D-glycopyranosyl]-β-D-glycopyranosyl]-β-D-glycopyranosyl]-β-D-glycopyranosyl]-β-D-glycopyranosyl]-β-D-glycopyranosyl]-β-D-glycopyranosyl]-α-D-glycopyranoside 82 (390 mg, 91%). [¹H NMR (400 MHz, CDCl₃) δ 8.10-7.08 (m, 75H), 6.57 (d, J=4.0 Hz, 1H), 5.88 (t, J=8.8 Hz, 1H), 5.73 (s, 1H), 5.70-5.50 (m, 5H), 5.40-5.09 (m, 5H), 4.95-4.65 (m, 9H), 4.55-4.31 (m, 6H), 4.20-3.30 (m, 34H), 2.58-2.30 (m, 3H), 2.01 (s, 3H), 1.98 (s, 3H), 1.95 (s, 3H), 1.89 (s, 3H), 1.88 (s, 3H), 1.63-1.55 (m, 6H), 1.01 (s, 3H), 0.90 (s, 3H), 0.87 (s, 3H), 0.87 (s, 3H), 0.82 (s, 3H), 0.65 (s, 3H).]

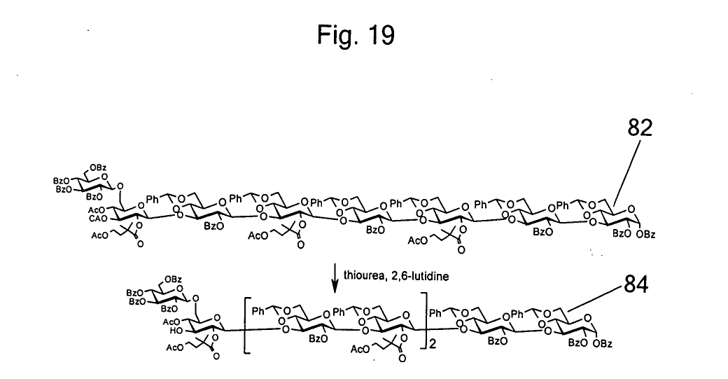

(f) FIG. 19

A mixture of Benzoyl 2-O-benzoyl-4,6-O-benzylidene-3-O-[2-O-benzoyl-4,6-O-benzylidene-3-O-[2-O-(4-acetoxy-2,2-dimethylbutanoyl)-4,6-O-benzylidene-3-O-[2-O-benzoyl-4,6-O-benzylidene-3-O-[2-O-(4-acetoxy-2,2-dimethylbutanoyl)-4,6-O-benzylidene-3-O-[2-O-benzoyl-4,6-O-benzylidene-3-O-[2-O-(4-acetoxy-2,2-dimethylbutanoyl)-4-O-Acetyl-3-O-chloroacetyl-6-O-[2,3,4,6-tetra-O-benzoyl-β-D-glycopyranosyl]-β-D-glycopyranosyl]-α-D-glycopyranosyl]-β-D-glycopyranosyl]-β-D-glycopyranosyl]-β-D-glycopyranosyl]-β-D-glycopyranosyl]-α-D-glycopyranoside 82 (153 mg, 0.047 mmol), thiourea (18 mg, 5 eq.), and 2,6-dimethylpyridine (5.5 μL, 1 eq.) in MeOH (4 mL) and CH₂Cl₂ (6 mL) was boiled under reflux overnight. The mixture was concentrated and the residue was extracted with CH₂Cl₂. The extract was washed successively with cold dilute HCl, aqueous NaHCO₃, and H₂O, dried, and concentrated. The residue was purified to by silica gel column chromatography to afford Benzoyl 2-O-benzoyl-4,6-O-benzylidene-3-O-[2-O-benzoyl-4,6-O-benzylidene-3-O-[2-O-(4-acetoxy-2,2-dimethylbutanoyl)-4,6-O-benzylidene-3-O-[2-O-benzoyl-4,6-O-benzylidene-3-O-[2-O-(4-acetoxy-2,2-dimethylbutanoyl)-4,6-O-benzylidene-3-O-[2-O-benzoyl-4,6-O-benzylidene-3-O-[2-O-(4-acetoxy-2,2-dimethylbutanoyl)-4-O-Acetyl-6-O-[2,3,4,6-tetra-O-benzoyl-β-D-glycopyranosyl]-β-D-glycopyranosyl]-β-D-glycopyranosyl]-β-D-glycopyranosyl]-β-D-glycopyranosyl]-β-D-glycopyranosyl]-β-D-glycopyranosyl]-α-D-glycopyranoside 84 (131 mg, 88%). [¹H NMR (400 MHz, CDCl₃) δ 8.10-7.08 (m, 75H), 6.57 (d, J=4.0 Hz, 1H), 5.88 (t, J=8.8 Hz, 1H), 5.73 (s, 1H), 5.70-5.50 (m, 4H), 5.40-5.09 (m, 5H), 4.95-4.65 (m, 9H), 4.55-4.31 (m, 6H), 4.20-3.30 (m, 33H), 2.58-2.30 (m, 3H), 2.01 (s, 3H), 1.98 (s, 3H), 1.95 (s, 3H), 1.89 (s, 3H), 1.88 (s, 3H), 1.63-1.55 (m, 6H), 1.01 (s, 3H), 0.90 (s, 3H), 0.87 (s, 3H), 0.87 (s, 3H), 0.82 (s, 3H), 0.65 (s, 3H); ¹³C NMR δ 175.94, 175.88, 175.80, 171.62, 171.07, 170.92, 170.55, 166.37, 166.02, 165.81, 165.40, 165.36, 164.98, 164.95, 164.60, 164.59 (16C, 16×C═O), 102.86, 102.73, 101.78, 101.77, 101.32, 101.32, 101.32, 101.06, 100.44, 100.25, 98.71, 96.25, 96.13, 90.50 (14C, 8×C₁ and 6×PhCH).]

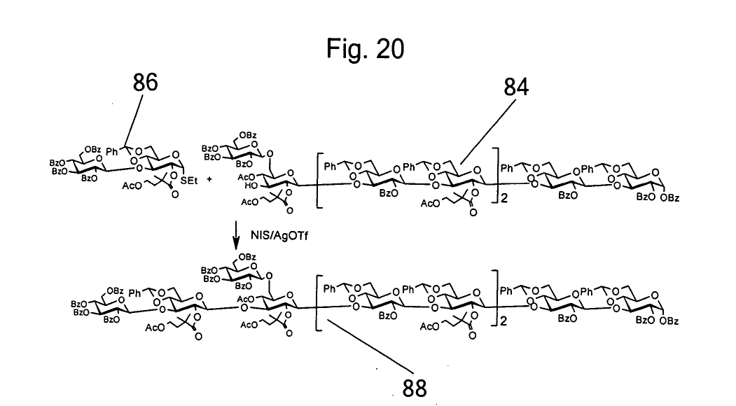

(g) FIG. 20

Thioglycoside 86 was prepared by condensation of 2,3,4,6-tetra-O-benzoyl-α-D-glucopyranosyl trichloroimidate 54 with Thioglycoside 2 (Ethyl 2-O-(4-acetoxy-2,2-dimethylbutanoyl)-4,6-O-benzylidene-1-thio-α-D-glycopyranoside) 40 in CH₂Cl₂ in the presence of trimethylsilyl triflate.

To a stirred mixture of octasaccharide Benzoyl 2-O-benzoyl-4,6-O-benzylidene-3-O-[2-O-benzoyl-4,6-O-benzylidene-3-O-[2-O-(4-acetoxy-2,2-dimethylbutanoyl)-4,6-O-benzylidene-3-O-[2-O-benzoyl-4,6-O-benzylidene-3-O-[2-O-(4-acetoxy-2,2-dimethylbutanoyl)-4,6-O-benzylidene-3-O-[2-O-benzoyl-4,6-O-benzylidene-3-O-[2-O-(4-acetoxy-2,2-dimethylbutanoyl)-4-O-Acetyl-6-O-[2,3,4,6-tetra-O-benzoyl-α-D-glycopyranosyl]-β-D-glycopyranosyl]-β-D-glycopyranosyl]-β-D-glycopyranosyl]-β-D-glycopyranosyl]-β-D-glycopyranosyl]-β-D-glycopyranosyl]-α-D-glycopyranoside 84 (100 mg, 0.03 mmol), thioglycoside 86 (63 mg, 1.9 eq.) and molecular sieves (4 Å, 100 mg) in CH₂Cl₂ (5 mL) at −20° C. were added NIS (15 mg) and AgOTf (2 mg in 0.3 mL dry toluene). After stirring for 10 h, the reaction mixture was quenched with Et₃N (0.1 mL), filtered, and the filtrate was concentrated in vacuo. The residue was purified by silica gel column chromatography to give Benzoyl 2-O-benzoyl-4,6-O-benzylidene-3-O-[2-O-benzoyl-4,6-O-benzylidene-3-O-[2-O-(4-acetoxy-2,2-dimethylbutanoyl)-4,6-O-benzylidene-3-O-[2-O-benzoyl-4,6-O-benzylidene-3-O-[2-O-(4-acetoxy-2,2-dimethylbutanoyl)-4,6-O-benzylidene-3-O-[2-O-benzoyl-4,6-O-benzylidene-3-O-[2-O-(4-acetoxy-2,2-dimethylbutanoyl)-4-O-Acetyl-6-O-[2,3,4,6-tetra-O-benzoyl-β-D-glycopyranosyl]-3-O-[2-O-(4-acetoxy-2,2-dimethylbutanoyl)-4,6-O-benzylidene-3-O-[2,3,4,6-tetra-O-benzoly-β-D-glycopyranosyl]-β-D-glycopyranosyl]-β-D-glycopyranosyl]-β-D-glycopyranosyl]-β-D-glycopyranosyl]-β-D-glycopyranosyl]-β-D-glycopyranosyl]-β-D-glycopyranosyl]-β-D-glycopyranosyl]-α-D-glycopyranoside 88 (100 mg, 88%).

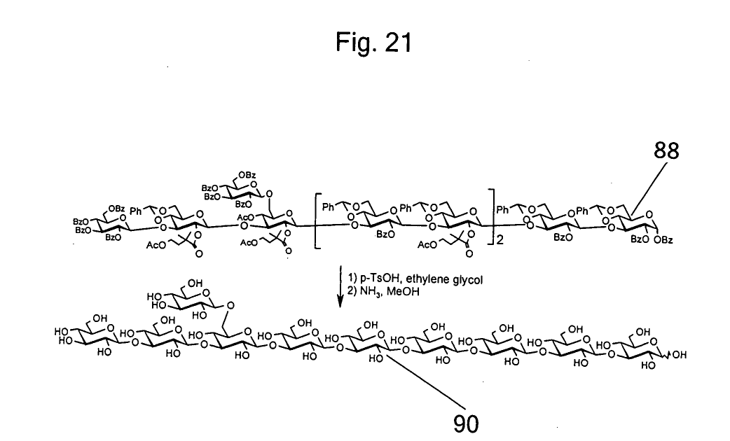

(h) FIG. 21

Compound Benzoyl 2-O-benzoyl-4,6-O-benzylidene-3-O-[2-O-benzoyl-4,6-O-benzylidene-3-O-[2-O-(4-acetoxy-2,2-dimethylbutanoyl)-4,6-O-benzylidene-3-O-[2-O-benzoyl-4,6-O-benzylidene-3-O-[2-O-(4-acetoxy-2,2-dimethylbutanoyl)-4,6-O-benzylidene-3-O-[2-O-benzoyl-4,6-O-benzylidene-3-O-[2-O-(4-acetoxy-2,2-dimethylbutanoyl)-4-O-Acetyl-6-O-[2,3,4,6-tetra-O-benzoyl-β-D-glycopyranosyl]-3-O-[2-O-(4-acetoxy-2,2-dimethylbutanoyl)-4,6-O-benzylidene-3-O-[2,3,4,6-tetra-O-benzoly-β-D-glycopyranosyl]-β-D-glycopyranosyl]-β-D-glycopyranosyl]-β-D-glycopyranosyl]-β-D-glycopyranosyl]-β-D-glycopyranosyl]-β-D-glycopyranosyl]-β-D-glycopyranosyl]-β-D-glycopyranosyl]-α-D-glycopyranoside 88 (0.13 g, 0.03 mmol) and ethylene glycol (0.2 mL) in CH₃CN (4 mL) was added p-toluenesulfonic acid (5 mg). The mixture was stirred overnight, neutralized with Et₃N and concentrated. After a short flash chromatography, the fractions were collected and concentrated. The residue was dissolved in 9 mL MeOH/NH₃, and stirred overnight. After concentration, the residue was purified by Bio-gel P2, the corresponding fractions were collected and freeze-dried to give amorphous branched decasaccharide 90 (44 mg, 89%). [¹H NMR (400 MHz, D₂O) δ 4.62 (d, J=8.0 Hz, 0.7H), 4.60 (d, J=8.0 Hz, 0.2H), 4.35 (d, J=8.0 Hz, 0.1H), 3.74 (bd, J=11.6 Hz, 1H), 3.62-3.43 (m, 2H), 3.37 (t, J=8.0 Hz, 1H), 3.35-3.25 (m, 2H).]

Binding of Synthetic Glucan Molecules to Glucan Receptor

Binding interactions between synthetic glucans and a recombinant Dectin receptor (kindly provided by Dr. Gordon Brown, Sir William Dunn School of Pathology, University of Oxford, Oxford, England) were investigated using a BIAcore 2000 surface plasmon resonance instrument (Biacore, Piscataway, N.J.) according to the methods of Kougias, Lowe, Rice and colleagues. Experiments were performed at 37° C. using a running buffer containing 150 mM NaCl, 10 mM HEPES, 3 mM EDTA and 0.005% polysorbate 20 (Biacore, Piscataway, N.Y.). Glucan phosphate was immobilized on three flowcells of a BIACORE CM5 sensor chip derivatized by exposure to EDC/NHS as reported by Kougias et al. The fourth flow cell served as a control surface without attached glucan phosphate. Competition studies using the immobilized glucan phosphate surface were performed at a flow rate of 20 μl/min. Recombinant Dectin was passed over the glucan surface for 300 sec, followed by sequential regeneration (60 sec exposures) with 0.3% triton X-100 and 3M guanidine HCl. Results were expressed as the mean ±SEM of >4 replicate experiments utilizing three flow cells.

The inventors demonstrated binding of synthetic glucan oligomers consisting of eight (octasaccharide), nine (nonasaccharide) and ten (decasaccharide) glucose subunits. All of the synthetic glucans produced by the method of the present invention were bound by recombinant Dectin in a specific, dose dependent and saturable manner.

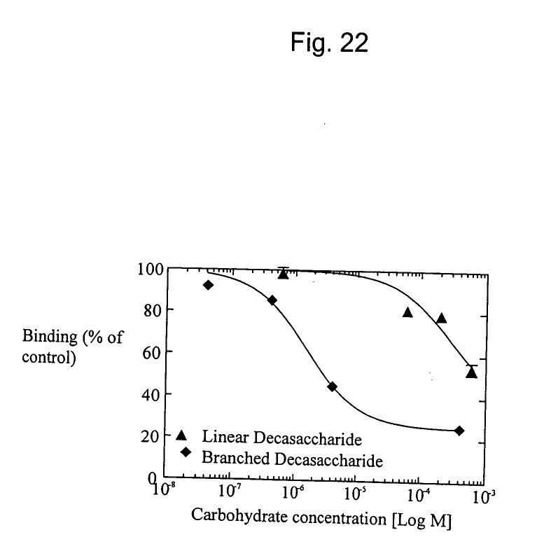

As demonstrated by the results shown graphically in FIG. 22, synthetic glucan polymers were generated which were linear (no side chain branches) or branched (single glucose subunit branch). Changing the basic structure of the polymer from linear to branched resulted in a 100-fold increase in binding affinity. Addition of a single glucose side chain branch increase the affinity of the receptor for the synthetic glucan. Affinity of the receptor for specific glucan structures increased as the size of the polymer increased (e.g.,. an unbranched decasaccharide was recognized with higher affinity than an unbranched nona-or octasaccharide), and a branched glucan polymer was recognized by its cognate receptor with higher affinity than a linear glucan polymer.

### REFERENCES

Kougias, P., et al. Normal Human Fibroblasts Express Pattern Recognition Receptors for Fungal (13)-β-D-Glucans. Infec. Immun. (2001) 69:3933-3938.
Lowe, E. P., et al. Human Vascular Endothelial Cells Express Pattern Recognition Receptors for Fungal Glucans Which Stimulates Nuclear Factor KB Activation and Interleukin 8 Production. Amer Surg (2002) 68:508-517.
Lowe, E., P. A (1-3)-β-D-linked heptasaccharide is the unit ligand for glucan pattern recognition receptors on human monocytes. Microbes and Infection (2001) 3:789-797.
Rice, P. J., et al. Human monocyte scavenger receptors are pattern recognition receptors for (1-3)-β-D-glcuans. Leuk Biol (2002) 72:140-146.

What is claimed is:

1. A method for forming β-linkages between units of a carbohydrate polymer, the method comprising attaching a protecting group chosen from 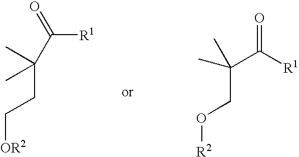
to a donor molecule, where R¹ activates ester formation and R² initiates lactone formation upon its removal from the donor.

2. The method of claim 1 wherein the protecting group is 4-acetoxy-2,2-dimethylbutanoyl chloride.

3. The method of claim 1 wherein the donor molecule is a glucoside donor.

4. The method of claim 1 wherein the protecting group is attached to the donor at the C2 position.

5. The method of claim 1 wherein R¹ is optionally substituted with Cl or anhydride.

6. The method of claim 1 wherein R² is optionally substituted with silane or acetate.

7. A method for forming a βglucan molecule, the method comprising attaching a C2-carboxy protecting group to a glucoside donor to prevent orthoester formation during attachment of carbohydrate units to form the β-glucan molecule.

8. The method of claim 7 wherein the protecting group is 4-acetoxy-2,2-dimethylbutanoyl chloride.

9. The method of claim 7 wherein the glucan is a β-1,3-D-glucan.

10. A carbohydrate polymer produced by the method of claim 1 or claim 7.

11. A pharmaceutical preparation comprising at least one β-1,3-D-glucan synthesized by the method of forming β-linkages between anhydroglucose molecules by attaching 4-acetoxy-2,2-dimethylbutanoyl chloride to a glucoside donor molecule prior to attachment of the donor to an acceptor molecule.
